(19) United States
(12) Patent Application Publication
(10) Pub. No.: US 2016/0018423 A1
(43) Pub. Date: Jan. 21, 2016

(54) NON-HIGH DENSITY LIPOPROTEIN DERIVED CVD MARKERS

(71) Applicant: ZORA BIOSCIENCES OY, Espoo (FI)

(72) Inventor: Reijo Laaksonen, Lempäälä (FI)

(21) Appl. No.: 14/773,095

(22) PCT Filed: Mar. 7, 2014
(86) PCT No.: PCT/EP2014/054499

§ 371 Date: Sep. 4, 2015

Related U.S. Application Data
(60) Provisional application No. 61/775,445, filed on Mar. 8, 2013.

(51) Int. Cl. G01N 33/92

(52) U.S. Cl. CPC G01N 33/92; G01N 2405/08; G01N 2405/02; G01N 2405/04; G01N 2800/323; G01N 2333/775; G01N 2570/00

(57) ABSTRACT

The present invention inter alia relates to methods and uses involving the determination of lipid/lipid concentration ratios in order to diagnose, predict, prevent and/or treat atherosclerosis or cardiovascular disease (CVD) and its complications including, e.g., acute myocardial infarction. The methods include analyzing lipid concentrations and resulting lipid/lipid concentration ratios of a non-high density lipoprotein samples from patients and comparing them to a control.

### FIELD OF THE INVENTION

The present invention relates to methods and uses involving the determination of lipid/lipid concentration ratios in order to diagnose, predict, prevent and/or treat atherosclerosis or cardiovascular disease (CVD) and its complications including, e.g., acute myocardial infarction or cardiovascular death. The methods include analyzing lipid concentrations and resulting lipid/lipid concentration ratios of a biological sample and comparing them to a control. Specifically, the current invention relates to the identification of novel lipid biomarkers that are superior CVD markers compared to standard lipid tests including plasma/serum total cholesterol, triglycerides, LDL-C, HDL-C, or ratios thereof. The novel biomarkers are ratios that are derived from concentrations of molecular lipid species or lipid class sums of triacylglycerides together with ceramides and ceramide derivatives (glucosyl/galactosyl ceramides, GM3 gangliosides), cholesteryl esters, or lysophospholipids.

### BACKGROUND OF THE INVENTION

Worldwide, cardiovascular diseases (CVD) are among the leading causes of mortality and morbidity with ever-increasing prevalence. CVD is used to classify numerous conditions that affect the heart, heart valves, blood, and vasculature of the body. One of these conditions is coronary artery disease (CAD). A central aspect in the development of CAD is the accumulation of lipid material in the blood vessel walls that could result in atherosclerotic plaques, complex molecular formations that contain numerous lipids. The main lipid sources are the low density lipoprotein (LDL) particles, which easily penetrate and get trapped in the arterial wall, where they are subjected to modifications (e.g., oxidation or aggregation) that enhance their retention. High density lipoprotein (HDL) particles, which are generally considered as anti-atherogenic, are able to remove LDL derived lipids from the vessel walls in a process called reverse cholesterol transport. Furthermore, HDL particles possess beneficial anti-inflammatory, anti-apoptotic and anti-oxidative properties.

It is generally accepted that high total cholesterol or low-density lipoprotein cholesterol (LDL-C) concentrations in the blood predict risk for CVD. Plasma or serum total cholesterol, LDL-C or HDL-C concentrations have been used as gold standard biomarkers for CVD/CAD risk prediction. Based on large scale population studies, it is evident that the standard cholesterol measurements associate with the CAD risk and CAD endpoints such as acute myocardial infarction (AMI) or cardiovascular death. Lowering of cholesterol concentrations, especially of LDL-C (mainly by statin treatment), has therefore been the main therapeutic strategy, which has successfully decreased the amount of incidences on the population level.

However, LDL-C may not be an optimal target for diagnostic/therapeutic purposes for several reasons. Firstly, it has been observed that one half of acute myocardial infarction (AMI) patients have LDL-cholesterol levels which are within the recommended normal range. Secondly, it is important to note that there is still a substantial (˜65%) residual risk of developing CVD/CAD in statin treated patients despite a lowering of LDL-C. Accordingly, there is a need for additional diagnostic/therapeutic targets beyond LDL-C.

Part of the residual risk is due to low HDL-C levels. Levels of apolipoprotein A1, the main surface protein on HDL particles, and HDL-C, the amount of cholesterol in those particles, correlate with each other and are considered separately as negative risk factors. The ratio of total cholesterol/HDL cholesterol, which is readily calculated from the results of standard lipid tests, is therefore another well-established marker in CVD risk assessment. Raising HDL-C levels, e.g., by using niacin, fibrates, or CETP-inhibitors, has been another therapeutic approach in combination with LDL-C lowering. In addition to total cholesterol, LDL-C, and HDL-C, several non-lipid risk factors (including age, blood pressure, diabetes, smoking, and body-mass-index) are used in risk assessment to evaluate/calculate an individual's risk for cardiovascular events. The best known risk score, the Framingham score, evaluates an individual's 10-year risk of having a cardiovascular event and is calculated from factors including total cholesterol and HDL-C, age, gender, blood pressure, smoking status, and the use of lipid-lowering medication. However, while these risk scores may be useful at the population level, they do not very accurately reveal an individual's risk for cardiovascular events.

Statins are a family of cholesterol lowering drugs for people at high risk of cardiovascular complications. Statins are widely used. For example, in the USA alone there are almost 20 million statin treated patients. Moreover, it has been calculated that some 50 million patients would benefit from statin treatment in the USA alone. However, despite statin treatment the CVD patients have a substantial risk of developing severe CVD complications. An early targeted initiation of preventive measures for CVD-related severe complications, such as AMI and death, would be of great benefit and would provide a major opportunity in reducing mortality and morbidity in patients suffering from CVD. Accurate identification of individuals who are at risk of developing CVD and CVD complications is essential. Traditional risk assessment fails to recognize a large proportion of patients at high risk, while a large proportion of individuals are classified as having intermediate risk, leaving patient management uncertain. Additional strategies to further refine risk assessment of high-risk CVD are therefore highly needed.

Both LDL and HDL are highly heterogeneous classes of particles; and both can be further divided into subclasses that differ in size, density, charge, as well as in their molecular composition. These properties are suggested to directly affect the atherogenic or anti-atherogenic potential of the lipoproteins: the ability to penetrate and get trapped in the arterial wall, or the ability to exert anti-oxidative and anti-inflammatory properties and to remove cholesterol from arterial walls. However, the link of particle subclass and pro/anti-atherogenic potential is not very well understood. For example, smaller LDL particles have been suggested to be more atherogenic than larger LDL particles, but contradictory evidence also exists.

In addition to LDL and HDL, human plasma or serum contains other triglyceride-rich particles. These include very-low density lipoproteins (VLDL) and intermediate-density lipoproteins (IDL). Total plasma/serum containing LDL, VLDL, and IDL particles are collectively called the non-HDL fraction. The non-HDL fraction is simply the fraction of total plasma/serum excluding the HDL fraction. The lipoproteins of the non-HDL fraction have a common origin and are considered to be functionally and compositionally related. VLDL, synthesized in the liver, is processed intra-vascularly by lipases and lipid exchange proteins, resulting in the formation of the intermediate product, IDL, which is further processed to LDL. LDL is the major constituent of the non-HDL fraction, followed by VLDL (whose mass is roughly one third that of LDL), while IDL is present as a minor species. In practice, LDL-C is often not directly measured but calculated using equations (e.g., the Friedewald equation), by subtracting HDL-C and triglyceride content (divided by 2.2) from total cholesterol content.

Different lipoprotein subclasses can differ dramatically in their lipid composition. As a consequence, two people with the same result from standard cholesterol tests can differ substantially in the amount and size of lipoprotein particles (FIG. 1). It has therefore been suggested that the particle numbers instead of cholesterol content would better reflect the risk of developing CVD. Apolipoprotein B (ApoB) is the main surface protein of LDL, VLDL, and IDL particles (non-HDL fraction), present as one ApoB molecule on each particle. ApoB levels therefore directly reflect the amount of atherogenic lipoprotein particles present in the sample, whereas the amount of particles and cardiovascular risk could vary substantially for a given LDL-C concentration when the particle size (and cholesterol content) varies. The distribution, size and particle numbers of a sample are easily obtained with Nuclear Magnetic Resonance (NMR) spectroscopy. Some studies, but not all, have found that the amount of LDL particles (LDL-P) or ApoB is more closely associated with CVD risk than LDL-C. However, recent studies have demonstrated that neither the determination of LDL-P nor the distribution of small and large particles improves CVD risk assessment obtained by calculating the total/HDL cholesterol ratio (Parish et al., 2012).

A major drawback of LDL-C, HDL-C, and other parameters that determine particle numbers is that they do not necessarily reflect the quality of the particles. Cholesterol is clearly associated with CVD, but despite the reputation of having a central role, no convincing evidence of cholesterol being the effective risk molecule seems to exist. One of the central arguments in favor of the role of cholesterol comes from the risk reduction related to statin use: taking cholesterol-lowering medication reduces the risk for CVD. However, this is a rough oversimplification, since statins are known to reduce levels of several other molecules besides cholesterol. As cholesterol is essentially present in all lipoprotein particles, it may only be an indirect measurement associated with other dangerous molecules carried by the LDL particle. Moreover, from a molecular point of view, cholesterol is present in lipoprotein particles in functionally distinct forms: as free cholesterol, or esterified to different fatty acids. Thus, the LDL-C and HDL-C tests that determine the total cholesterol (free and esterified) in these particular particles may not reflect the quality and function of the particles very accurately. These properties are likely determined by factors other than total cholesterol, such as the presence of bioactive protein or lipid molecules, which could have better diagnostic or prognostic value. The quality of lipoproteins is traditionally studied by biochemical methods, measuring, e.g., the tendency of LDL particles to get oxidized or aggregated. These methods are time-consuming and are not fit for routine clinical use. Therefore, robust methods which are able to measure the quality of lipoproteins and which are suitable for routine clinical use are required. Identifying which molecules directly relate to the function of the lipoproteins and hazardous or even fatal cardiovascular events is therefore highly important.

In contrast to protein constituents often present in particular lipoprotein classes, any given lipid molecule may be found (albeit in different amounts) in basically any lipoprotein (sub)class. Lipoprotein particles consist of a neutral core where hydrophobic lipids such as triacylglycerol (TAG) and cholesteryl ester (CE) are abundantly located, surrounded by a layer of structural proteins and polar lipids, including phospholipids and sphingolipids. The minor lipid components located at the lipoprotein surface include highly potent bioactive ceramides (Cer, Glc/GalCer), GM3 gangliosides, and lysophospholipids, increased plasma levels of which have been linked to metabolic diseases including diabetes and atherosclerosis. Increased plasma TAG levels are similarly considered to associate with cardiovascular risk. In the circulation, the majority of TAG is transported mainly in VLDL and IDL particles, and less is known about the role of these lipids in the LDL fraction.

Since lipoprotein (sub)classes have different functional roles, it is relevant to assess with which particle a specific lipid molecule is associated. The distribution of the lipid between harmful and beneficial particles could therefore be affected without a change in its circulating levels, and the enrichment of relevant lipid molecules in certain particles may be masked if measured from total plasma/serum. As an example, cholesterol is sometimes called “good” or “bad” depending on whether it is in HDL or LDL fractions, respectively. As mentioned above, however, LDL-C is not an optimal biomarker for CVD diagnostic and therapeutic purposes.

There is a need in the art for novel biomarkers that are indicative of atherosclerosis or CVD and its complications, or the risk therefore, which biomarkers are alternatives or superior to standard lipid tests including plasma/serum total cholesterol, triglycerides, LDL-C, HDL-C, or ratios thereof.

### SUMMARY OF THE INVENTION

The present invention inter alia provides novel biomarkers and associated diagnostic methods and uses for the identification of subjects suffering from atherosclerosis or CVD, and/or one or more of the complications of CVD, or being at risk of developing atherosclerosis or CVD, and/or one or more of the complications of CVD. Such methods and uses comprise monitoring specific lipid/lipid concentration ratios from a non-HDL sample from such a subject, such as the LDL fraction of the subject's plasma or serum, and comparing such ratios to those in a control.

Accordingly, in a first aspect of the invention, a method is provided for determining whether a subject is at risk to develop, or is suffering from atherosclerosis or cardiovascular disease (CVD) and/or one or more of its complications, said method comprising determining in a non-HDL sample from said subject a ceramide/TAG concentration ratio, wherein a decreased ratio in said sample, when compared to a control, is indicative of said subject suffering from or having an increased risk of developing atherosclerosis or CVD and/or one or more of its complications.

In a preferred embodiment, the ceramide/TAG concentration ratio whose decrease is compared to the control is selected from any of the ceramide/TAG concentration ratios referred to in Table 1.

According to a particularly preferred embodiment, the ceramide/TAG concentration ratio whose decrease is compared to the control is selected from: Glc/GalCer(d18:1/22:0)/TAG 50:1 total (16:0/16:0/18:1), Cer(d18:1/22:0)/TAG 50:1 total (16:0/16:0/18:1), and GM3-d18:1/24:0/TAG 54:2 total (18:0/18:1/18:1).

In another embodiment of this aspect of the invention, a method is provided for determining whether a subject is at risk to develop, or is suffering from atherosclerosis or cardiovascular disease (CVD) and/or one or more of its complications, said method comprising determining in a non-HDL sample from said subject a CE/TAG concentration ratio, wherein a decreased ratio in said sample, when compared to a control, is indicative of said subject suffering from or having an increased risk of developing atherosclerosis or CVD and/or one or more of its complications.

In a preferred embodiment, the CE/TAG concentration ratio whose decrease is compared to the control is selected from any of the CE/TAG concentration ratios referred to in Table 2.

According to a particularly preferred embodiment, the CE/TAG concentration ratio whose decrease is compared to the control is selected from: CE 22:6/TAG 50:1 total (16:0/16:0/18:1), CE 16:0/TAG 50:1 total (16:0/16:0/18:1), and CE 22:6/TAG 50:2 total (14:0/18:1/18:1)(16:0/16:0/18:2)(16:0/16:1/18:1).

In yet another embodiment of this aspect of the invention, a method is provided for determining whether a subject is at risk to develop, or is suffering from atherosclerosis or cardiovascular disease (CVD) and/or one or more of its complications, said method comprising determining in a non-HDL sample from said subject a LPE/TAG concentration ratio, wherein a decreased ratio in said sample, when compared to a control, is indicative of said subject suffering from or having an increased risk of developing atherosclerosis or CVD and/or one or more of its complications.

In a preferred embodiment, the LPE/TAG concentration ratio whose decrease is compared to the control is selected from any of the LPE/TAG concentration ratios referred to in Table 3.

According to a particularly preferred embodiment, the LPE/TAG concentration ratio whose decrease is compared to the control is selected from: LPE 18:0/TAG 50:1 total (16:0/16:0/18:1), LPE 18:0/TAG 50:2 total (14:0/18:1/18:1)(16:0/16:0/18:2)(16:0/16:1/18:1), and LPE 16:0/TAG 50:2 total (14:0/18:1/18:1)(16:0/16:0/18:2)(16:0/16:1/18:1).

In yet another embodiment of this aspect of the invention, a method is provided for determining whether a subject is at risk to develop, or is suffering from atherosclerosis or cardiovascular disease (CVD) and/or one or more of its complications, said method comprising determining in a non-HDL sample from said subject a ceramide/CE concentration ratio, wherein an decreased ratio in said sample, when compared to a control, is indicative of said subject suffering from or having an increased risk of developing atherosclerosis or CVD and/or one or more of its complications.

In a preferred embodiment, the ceramide/CE concentration ratio whose decrease is compared to the control is selected from any of the ceramide/CE concentration ratios referred to in Table 4.

According to a particularly preferred embodiment, the ceramide/CE concentration ratio whose increase is compared to the control is selected from: Cer(d18:1/18:0)/CE 22:0, Cer(d18:1/20:0)/CE 22:0, and Cer(d18:1/22:0)/CE 22:0.

In another aspect of the present invention, a method is provided for obtaining data for use in determining whether a subject is at risk to develop, or is suffering from atherosclerosis or cardiovascular disease (CVD) and/or one or more of its complications, said method comprising determining in a non-HDL sample from said subject a ceramide/TAG concentration ratio.

In a preferred embodiment, the ceramide/TAG concentration ratio is selected from any of the ceramide/TAG concentration ratios referred to in Table 1.

According to a particularly preferred embodiment, the ceramide/TAG concentration ratio is selected from: Glc/GalCer(d18:1/22:0)/TAG 50:1 total (16:0/16:0/18:1), Cer(d18:1/22:0)/TAG 50:1 total (16:0/16:0/18:1), and GM3-d18:1/24:0/TAG 54:2 total (18:0/18:1/18:1).

In another embodiment of this aspect of the invention, a method is provided for obtaining data for use in determining whether a subject is at risk to develop, or is suffering from atherosclerosis or cardiovascular disease (CVD) and/or one or more of its complications, said method comprising determining in a non-HDL sample from said subject a CE/TAG concentration ratio.

In a preferred embodiment, the CE/TAG concentration ratio is selected from any of the CE/TAG concentration ratios referred to in Table 2.

According to a particularly preferred embodiment, the CE/TAG concentration ratio is selected from: CE 22:6/TAG 50:1 total (16:0/16:0/18:1), CE 16:0/TAG 50:1 total (16:0/16:0/18:1), and CE 22:6/TAG 50:2 total (14:0/18:1/18:1)(16:0/16:0/18:2)(16:0/16:1/18:1).

In yet another embodiment of this aspect of the invention, a method is provided for obtaining data for use in determining whether a subject is at risk to develop, or is suffering from atherosclerosis or cardiovascular disease (CVD) and/or one or more of its complications, said method comprising determining in a non-HDL sample from said subject a LPE/TAG concentration ratio.

In a preferred embodiment, the LPE/TAG concentration ratio is selected from any of the LPE/TAG concentration ratios referred to in Table 3.

According to a particularly preferred embodiment, the LPE/TAG concentration ratio is selected from: LPE 18:0/TAG 50:1 total (16:0/16:0/18:1), LPE 18:0/TAG 50:2 total (14:0/18:1/18:1)(16:0/16:0/18:2)(16:0/16:1/18:1), and LPE 16:0/TAG 50:2 total (14:0/18:1/18:1)(16:0/16:0/18:2)(16:0/16:1/18:1).

In yet another embodiment of this aspect of the invention, a method is provided for obtaining data for use in determining whether a subject is at risk to develop, or is suffering from atherosclerosis or cardiovascular disease (CVD) and/or one or more of its complications, said method comprising determining in a non-HDL sample from said subject a ceramide/CE concentration ratio.

In a preferred embodiment, the ceramide/CE concentration ratio is selected from any of the ceramide/CE concentration ratios referred to in Table 4.

According to a particularly preferred embodiment, the ceramide/CE concentration ratio is selected from: Cer(d18:1/18:0)/CE 22:0, Cer(d18:1/20:0)/CE 22:0, and Cer(d18:1/22:0)/CE 22:0.

In another aspect of the present invention, a method is provided for evaluating the effectiveness of a treatment of atherosclerosis or CVD and/or one or more of its complications in a subject, comprising determining in a non-HDL sample from said subject a ceramide/TAG concentration ratio, wherein a decreased ratio in said sample, when compared to a control, is indicative of the effectiveness of said treatment.

In a preferred embodiment, the ceramide/TAG concentration ratio whose decrease is compared to the control is selected from any of the ceramide/TAG concentration ratios referred to in Table 1.

According to a particularly preferred embodiment, the ceramide/TAG concentration ratio whose decrease is compared to the control is selected from: Glc/GalCer(d18:1/22:0)/TAG 50:1 total (16:0/16:0/18:1), Cer(d18:1/22:0)/TAG 50:1 total (16:0/16:0/18:1), and GM3-d18:1/24:0/TAG 54:2 total (18:0/18:1/18:1).

In another embodiment of this aspect of the invention, a method is provided for evaluating the effectiveness of a treatment of atherosclerosis or CVD and/or one or more of its complications in a subject, comprising determining in a non-HDL sample from said subject a CE/TAG concentration ratio, wherein a decreased ratio in said sample, when compared to a control, is indicative of the effectiveness of said treatment.

In a preferred embodiment, the CE/TAG concentration ratio whose decrease is compared to the control is selected from any of the CE/TAG concentration ratios referred to in Table 2.

According to a particularly preferred embodiment, the CE/TAG concentration ratio whose decrease is compared to the control is selected from: CE 22:6/TAG 50:1 total (16:0/16:0/18:1), CE 16:0/TAG 50:1 total (16:0/16:0/18:1), and CE 22:6/TAG 50:2 total (14:0/18:1/18:1)(16:0/16:0/18:2)(16:0/16:1/18:1).

In yet another embodiment of this aspect of the invention, a method is provided for evaluating the effectiveness of a treatment of atherosclerosis or CVD and/or one or more of its complications in a subject, comprising determining in a non-HDL sample from said subject a LPE/TAG concentration ratio, wherein a decreased ratio in said sample, when compared to a control, is indicative of the effectiveness of said treatment.

In a preferred embodiment, the LPE/TAG concentration ratio whose decrease is compared to the control is selected from any of the LPE/TAG concentration ratios referred to in Table 3.

According to a particularly preferred embodiment, the LPE/TAG concentration ratio whose decrease is compared to the control is selected from: LPE 18:0/TAG 50:1 total (16:0/16:0/18:1), LPE 18:0/TAG 50:2 total (14:0/18:1/18:1)(16:0/16:0/18:2)(16:0/16:1/18:1), and LPE 16:0/TAG 50:2 total (14:0/18:1/18:1)(16:0/16:0/18:2)(16:0/16:1/18:1).

In yet another embodiment of this aspect of the invention, a method is provided for evaluating the effectiveness of a treatment of atherosclerosis or CVD and/or one or more of its complications in a subject, comprising determining in a non-HDL sample from said subject a ceramide/CE concentration ratio, wherein an decreased ratio in said sample, when compared to a control, is indicative of the effectiveness of said treatment.

In a preferred embodiment, the ceramide/CE concentration ratio whose decrease is compared to the control is selected from any of the ceramide/CE concentration ratios referred to in Table 4.

According to a particularly preferred embodiment, the ceramide/CE concentration ratio whose increase is compared to the control is selected from: Cer(d18:1/18:0)/CE 22:0, Cer(d18:1/20:0)/CE 22:0, and Cer(d18:1/22:0)/CE 22:0.

In another preferred embodiment, the method for evaluating the effectiveness of a treatment of atherosclerosis or CVD and/or one or more of its complications in a subject according to the invention may further comprise after the determining step, changing, supplementing, or keeping the same an already administered treatment in said subject based on the ceramide/TAG concentration ratio, CE/TAG concentration ratio, LPE/TAG concentration ratio, or ceramide/CE concentration ratio obtained in the determining step.

In another aspect of the present invention, a method is provided for choosing an appropriate treatment of atherosclerosis or CVD and/or one or more of its complications in a subject, comprising determining in a non-HDL sample from said subject a ceramide/TAG concentration ratio, wherein a decreased ratio in said sample, when compared to a control, is indicative of said subject being in need of treatment or a change in, or supplementation of, an already administered treatment.

In a preferred embodiment, the ceramide/TAG concentration ratio whose decrease is compared to the control is selected from any of the ceramide/TAG concentration ratios referred to in Table 1.

According to a particularly preferred embodiment, the ceramide/TAG concentration ratio whose decrease is compared to the control is selected from: Glc/GalCer(d18:1/22:0)/TAG 50:1 total (16:0/16:0/18:1), Cer(d18:1/22:0)/TAG 50:1 total (16:0/16:0/18:1), and GM3-d18:1/24:0/TAG 54:2 total (18:0/18:1/18:1).

In another embodiment of this aspect of the invention, a method is provided for choosing an appropriate treatment of atherosclerosis or CVD and/or one or more of its complications in a subject, comprising determining in a non-HDL sample from said subject a CE/TAG concentration ratio, wherein a decreased ratio in said sample, when compared to a control, is indicative of said subject being in need of treatment or a change in, or supplementation of, an already administered treatment.

In a preferred embodiment, the CE/TAG concentration ratio whose decrease is compared to the control is selected from any of the CE/TAG concentration ratios referred to in Table 2.

According to a particularly preferred embodiment, the CE/TAG concentration ratio whose decrease is compared to the control is selected from: CE 22:6/TAG 50:1 total (16:0/16:0/18:1), CE 16:0/TAG 50:1 total (16:0/16:0/18:1), and CE 22:6/TAG 50:2 total (14:0/18:1/18:1)(16:0/16:0/18:2)(16:0/16:1/18:1).

In yet another embodiment of this aspect of the invention, a method is provided for choosing an appropriate treatment of atherosclerosis or CVD and/or one or more of its complications in a subject, comprising determining in a non-HDL sample from said subject a LPE/TAG concentration ratio, wherein a decreased ratio in said sample, when compared to a control, is indicative of said subject being in need of treatment or a change in, or supplementation of, an already administered treatment.

In a preferred embodiment, the LPE/TAG concentration ratio whose decrease is compared to the control is selected from any of the LPE/TAG concentration ratios referred to in Table 3.

According to a particularly preferred embodiment, the LPE/TAG concentration ratio whose decrease is compared to the control is selected from: LPE 18:0/TAG 50:1 total (16:0/16:0/18:1), LPE 18:0/TAG 50:2 total (14:0/18:1/18:1)(16:0/16:0/18:2)(16:0/16:1/18:1), and LPE 16:0/TAG 50:2 total (14:0/18:1/18:1)(16:0/16:0/18:2)(16:0/16:1/18:1).

In yet another embodiment of this aspect of the invention, a method is provided for choosing an appropriate treatment of atherosclerosis or CVD and/or one or more of its complications in a subject, comprising determining in a non-HDL sample from said subject a ceramide/CE concentration ratio, wherein an decreased ratio in said sample, when compared to a control, is indicative of said subject being in need of treatment or a change in, or supplementation of, an already administered treatment.

In a preferred embodiment, the ceramide/CE concentration ratio whose decrease is compared to the control is selected from any of the ceramide/CE concentration ratios referred to in Table 4.

According to a particularly preferred embodiment, the ceramide/CE concentration ratio whose increase is compared to the control is selected from: Cer(d18:1/18:0)/CE 22:0, Cer(d18:1/20:0)/CE 22:0, and Cer(d18:1/22:0)/CE 22:0.

In another preferred embodiment, the method provided for choosing an appropriate treatment of atherosclerosis or CVD and/or one or more of its complications in a subject may further comprise after the determining step, treating said subject based on the ceramide/TAG concentration ratio, CE/TAG concentration ratio, LPE/TAG concentration ratio, or ceramide/CE concentration ratio obtained in the determining step.

In accordance with all aspects and embodiments of the invention, the methods provided may be computer-implemented.

In a preferred embodiment, any of the computer-implemented methods of the invention may further comprise the steps of (i) obtaining by at least one processor information reflecting the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample; (ii) determining by at least one processor the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample; and (iii) outputting in user readable format the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample.

In another preferred embodiment of the computer-implemented methods of the invention, the methods may additionally further comprise the steps of (iv) determining by at least one processor a percentage difference between a control and the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample; and (v) outputting in user readable format the percentage difference obtained in the determining step (iv).

In a particularly preferred embodiment of the computer-implemented methods of the invention, the methods may additionally further comprise determining whether a subject is at risk to develop, or is suffering from atherosclerosis or cardiovascular disease (CVD) and/or one or more of its complications based on the percentage difference obtained in the outputting step.

In a preferred embodiment of the computer-implemented methods, the ceramide/TAG concentration ratio is selected from any of the ceramide/TAG concentration ratios referred to in Table 1, the CE/TAG concentration ratio is selected from any of the CE/TAG concentration ratios referred to in Table 2, the LPE/TAG concentration ratio is selected from any of the LPE/TAG concentration ratios referred to in Table 3, and the ceramide/CE concentration ratio is selected from any of the ceramide/CE concentration ratios referred to in Table 4.

According to a particularly preferred embodiment of the computer-implemented methods, the ceramide/TAG concentration ratio is selected from: Glc/GalCer(d18:1/22:0)/TAG 50:1 total (16:0/16:0/18:1), Cer(d18:1/22:0)/TAG 50:1 total (16:0/16:0/18:1), and GM3-d18:1/24:0/TAG 54:2 total (18:0/18:1/18:1); the CE/TAG concentration ratio is selected from: CE 22:6/TAG 50:1 total (16:0/16:0/18:1), CE 16:0/TAG 50:1 total (16:0/16:0/18:1), and CE 22:6/TAG 50:2 total (14:0/18:1/18:1)(16:0/16:0/18:2)(16:0/16:1/18:1); the LPE/TAG concentration ratio is selected from: LPE 18:0/TAG 50:1 total (16:0/16:0/18:1), LPE 18:0/TAG 50:2 total (14:0/18:1/18:1)(16:0/16:0/18:2)(16:0/16:1/18:1), and LPE 16:0/TAG 50:2 total (14:0/18:1/18:1)(16:0/16:0/18:2)(16:0/16:1/18:1); and the ceramide/CE concentration ratio is selected from: Cer(d18:1/18:0)/CE 22:0, Cer(d18:1/20:0)/CE 22:0, and Cer(d18:1/22:0)/CE 22:0.

Likewise in accordance with all aspects and embodiments of the invention, it is possible, and may be advantageous to determine at least 2 of the lipid/lipid concentration ratios referred to in respect of the respective aspect or embodiment. It is likewise preferred to determine at least 3, at least 4, at least 5, at least 6, at least 7, or at least 8 of the lipid/lipid concentration ratios referred to in respect of the respective aspect or embodiment.

In accordance with the invention, CVD is a disease characterized by coronary artery disease, peripheral artery disease, stroke and/or CVD death. It may or may not be atherosclerosis-induced.

The subject whose non-HDL sample is analyzed in connection with the methods and uses of the invention may be a subject having atherosclerosis. Alternatively, samples of subjects who do not have atherosclerosis may likewise be analyzed in accordance with the methods and uses of the invention.

It will be appreciated that it may be useful and even advantageous for the methods and uses of the invention to further comprise a step of determining the serum level of total cholesterol, low-density lipoprotein cholesterol (LDL-C), high-density lipoprotein cholesterol (HDL-C), Apolipoprotein B (ApoB) and/or Apolipoprotein C-III (ApoC-III) in a sample from said subject. Furthermore, according to a preferred embodiment of the methods or uses of the invention, the subject is preferably one that does not have elevated serum levels of one or more of total cholesterol, low-density lipoprotein cholesterol (LDL-C), Apolipoprotein C-III (ApoC-III) or Apolipoprotein B (ApoB), or a decreased serum level of HDL-cholesterol (HDL-C).

According to any of the methods or uses of the invention, the subject is being or has been treated with a statin, another lipid lowering drug, and/or a modulator of lipid/lipid concentration ratios. Alternatively, the subject may also be one that has not yet undergone statin therapy, therapy with another lipid lowering drug, and/or therapy with a modulator of lipid/lipid concentration ratios.

The non-HDL sample to be analyzed in accordance with the invention can advantageously be a plasma or serum sample which is substantially reduced in, essentially free of, or completely free of HDL particles (i.e., plasma or serum minus HDL). Also preferred is an embodiment where the non-HDL sample to be analyzed is an LDL sample. In another suitable embodiment, however, the non-HDL sample may also be a very-low density lipoprotein (VLDL) sample. It may furthermore advantageously be an intermediate-density lipoprotein (IDL) sample. In a still further embodiment, the non-HDL sample can advantageously be a combination of an LDL sample and a VLDL sample. A combination of an LDL sample and an IDL sample or a combination of a VLDL sample and an IDL sample are likewise suitable. Also preferred as a non-HDL sample of the invention is a combination of an LDL sample, a VLDL sample and an IDL sample.

In one aspect of the invention, a drug is provided which is capable of modulating a lipid/lipid concentration ratio according to the invention, for use in treating or preventing atherosclerosis or CVD and/or one or more of its complications. In one embodiment, the drug is administered such that said lipid/lipid concentration ratio in a sample from said subject does not markedly differ when compared to a control. In a preferred embodiment, the drug is a statin. It may, however, also advantageously be another lipid lowering drug. Alternatively, suitable as said drug is also a modulator of lipid/lipid concentration ratios, specifically the lipid/lipid concentration ratios described and/or claimed herein.

Accordingly, in another aspect the invention provides a method of treating or preventing atherosclerosis or CVD and/or one or more of its complications, in a subject in need thereof, comprising administering a therapeutically effective dose of a drug capable of modulating a lipid/lipid concentration ratio described and/or claimed herein. Again, the drug is suitably administered such that said lipid/lipid concentration ratio does not markedly differ when compared to a control. The drug is preferably a statin, although the use of another lipid lowering drug, or of a modulator of lipid/lipid concentration ratios is likewise contemplated.

In yet another aspect the invention provides a method of treating or preventing atherosclerosis or CVD and/or one or more of its complications in a subject in need thereof, comprising administering to the subject a therapeutically effective dose of a drug, wherein the drug is a statin; another lipid lowering drug selected from an HMG-CoA reductase inhibitor other than a statin, niacin (nicotinic acid), a cholesterol absorption inhibitor, a cholesteryl ester transfer protein (CETP), a bile acid sequestrant, a fibrate, a phytosterol, and a PCSK9 inhibitor; or a modulator of lipid/lipid concentration ratios selected from a small molecule, an antibody, an antisense RNA, a small interfering RNA (siRNA), and a natural or modified lipid, and wherein before administering the drug the subject has been identified as suffering from or having an increased risk of developing atherosclerosis or CVD and/or one or more of its complications based on a decreased ceramide/TAG concentration ratio, a decreased CE/TAG concentration ratio, a decreased LPE/TAG concentration ratio, or a decreased ceramide/CE concentration ratio as compared to a control.

In another aspect of the invention, a kit is provided for predicting or detecting atherosclerosis or CVD and/or one or more of its complications in a subject, or for performing any of the methods or uses according to the invention, wherein the kit comprises: (a) one or more lipid standards chosen from the lipids in any one of the lipid/lipid concentration ratios referred to in Tables 1 to 4; and/or (a) calibration line control(s); and/or positive and/or negative controls. Optionally, the kit of this aspect of the invention may further comprise one or more of the following components: (b) one or more control markers, such as a lipid or lipids, e.g., a lipid of any one of the lipid/lipid concentration ratios referred to in Tables 1 to 4, or a protein; (c) internal and/or external standards; (d) an agent, optionally an antibody, capable of binding any one of the lipids in any one of the lipid/lipid concentration ratios referred to in Tables 1 to 4; and (e) (a) reagent(s) for performing said methods or uses.

Another aspect of the invention relates to the use of a kit of the invention for predicting or detecting atherosclerosis or CVD and/or one or more of its complications, wherein the lipid/lipid concentration ratio in a sample from a subject is optionally determined by using mass spectrometry.

In another aspect of the invention, a kit is provided for predicting or detecting atherosclerosis or CVD and/or one or more of its complications in a subject, or for performing any of the methods or uses according to the invention, wherein the kit comprises: (a) an antibody or antibodies capable of binding any one of the lipids in any one of the lipid/lipid concentration ratios referred to in Tables 1 to 4 conjugated to an enzyme or a detectable label; or any one of the lipid(s) in any one of the lipid/lipid concentration ratios referred to in Tables 1 to 4 conjugated to an enzyme or a detectable label. The kit according this aspect of the invention may optionally further comprise one or more of the following components: (b) a substrate specific for said enzyme; (c) an assay plate coated with (a) secondary antibody(ies) capable of binding any of the antibodies of (a); (d) (a) standard(s) and/or (a) calibration line standard(s); (e) a stop solution and (f) necessary buffers and/or reagents required to perform the assay.

In a further embodiment, the kit of the invention comprises an enzyme conjugated to a protein which is specific to the detectable label on the antibody in (a) of the above embodiment, e.g. alkaline phosphatase conjugated to streptavidin.

In a preferred embodiment of this aspect of the invention, the kit of the above embodiment comprises an antibody conjugated to an enzyme or a detectable label which is capable of binding to any one of the lipids in the lipid/lipid concentration ratios referred to in the items or claims listed herein, such as item 1 or claim 1, and particularly item 7 or claim 14; and/or wherein the lipid conjugated to an enzyme or a detectable label is any one of the lipids in the lipid/lipid concentration ratios referred to in the items or claims listed herein, such as in item 1 or claim 1; and particularly item 7 or claim 14.

All kits of the invention may be accompanied by instructions to use them for predicting, diagnosing, or detecting atherosclerosis or CVD and/or one or more of its complications as defined herein.

In another aspect, the invention relates to an antibody against any one of the lipids in any one of the lipid/lipid concentration ratios referred to in Tables 1 to 4, for use in a) predicting a risk of a subject to develop, or to suffer from atherosclerosis or CVD and/or one or more of its complications; or b) preventing or treating atherosclerosis or CVD and/or one or more of its complications in a subject. Corresponding methods of a) predicting a risk of a subject to develop, or to suffer from atherosclerosis or CVD and/or one or more of its complications; or b) preventing or treating atherosclerosis or CVD, wherein such antibody is used as well as corresponding uses of such antibody are likewise embodiments of this aspect of the invention.

In one aspect, the invention relates to a statin, another lipid lowering drug, or a modulator of lipid/lipid concentration ratios for use for preventing or treating atherosclerosis or CVD and/or one or more of its complications in a subject, wherein said subject would be identified as being at risk to develop, or as suffering from atherosclerosis or CVD and/or one or more of its complications when applying any of the methods, drugs, kits, uses, or antibodies described and/or claimed herein.

In a further embodiment of this aspect, the invention relates to a statin, another lipid lowering drug, or a modulator of lipid/lipid concentration ratios for use for preventing or treating atherosclerosis or CVD and/or one or more of its complications in a subject, wherein said subject has been identified as being at risk to develop, or as suffering from atherosclerosis or CVD and/or one or more of its complications by any of the methods, drugs, kits, uses, or antibodies described and/or claimed herein.

In yet a further embodiment of this aspect, the invention relates to a statin, another lipid lowering drug, or a modulator of lipid/lipid concentration ratios for use for preventing or treating atherosclerosis or CVD and/or one or more of its complications in a subject, wherein said subject would be identified as not being at risk to develop, or as suffering from atherosclerosis or CVD and/or one or more of its complications by any of the methods, drugs, kits, uses, or antibodies described and/or claimed herein.

In yet a further embodiment of this aspect, the invention relates to a statin, another lipid lowering drug, or a modulator of lipid/lipid concentration ratios for use for preventing or treating atherosclerosis or CVD and/or one or more of its complications in a subject, wherein said subject has been identified as not being at risk to develop, or as suffering from atherosclerosis or CVD and/or one or more of its complications by any of the methods, drugs, kits, uses, or antibodies described and/or claimed herein.

Corresponding methods of preventing or treating atherosclerosis or CVD and/or one or more of its complications in a subject by administering a therapeutically effective amount of said statin, said other lipid lowering drug, or said modulator of lipid/lipid concentration ratios are likewise contemplated in accordance with this aspect of the invention, as are corresponding uses of said statin, said other lipid lowering drug, or said modulator of lipid/lipid concentration ratios.

In the context of the invention, the determination of lipid concentration(s) or lipid/lipid concentration ratio(s) is typically performed using an assay. Preferably, such assay is, or involves, mass spectrometry, nuclear magnetic resonance spectroscopy, fluorescence spectroscopy or dual polarisation interferometry, a high performance separation method such as HPLC, UHPLC or UPLC, an immunoassay such as an ELISA and/or an assay with a binding moiety capable of specifically binding the analyte. Particularly preferred is the use of mass spectrometry.

In the context of the invention, CVD includes endothelial dysfunction, coronary artery disease, angina pectoris, myocardial infarction, atherosclerosis, congestive heart failure, hypertension, cerebrovascular disease, stroke, transient ischemic attacks, deep vein thrombosis, peripheral artery disease, cardiomyopathy, arrhythmias, aortic stenosis, and aneurysm. Such diseases frequently involve atherosclerosis. In a preferred embodiment of the invention, the cardiovascular disease is a cardiovascular disease associated with atherosclerosis. In a further embodiment, CVD complications comprise, but are not limited to, myocardial infarcation (MI), angina pectoris, transischemic attack (TIA), stroke and death.

### BRIEF DESCRIPTION OF THE FIGURES

FIG. 1: The relationship between particle size and cholesterol content. Two examples of the particle composition in situations with identical LDL-C concentration demonstrating that, depending on the average particle size, the number of particles may differ drastically.

FIG. 2: Standard lipid tests fail to identify a marked proportion of CVD patients from healthy controls. A. The standard lipid tests are highlighted in the bottom box (dashed) of the volcano plot, where average percentage change between CVD patients (“case”) and controls are plotted against the statistical significance (p value). Each symbol represents a measured analyte (lipid or lipid/lipid ratio) either from serum or LDL fraction. B. Depiction of the concentrations (g/I or mmol/I) of clinical measurements of “cases” (squares) and controls (circles).

FIG. 3: A volcano plot representing the average percentage change in calculated LDL lipid/lipid ratios between cases and controls plotted against statistical significance (p value). Each symbol represents a calculated lipid/lipid ratio. The boxes highlight analytes or lipid ratios with no statistically significant difference (lower box) or with a highly significant difference between the study groups (upper box). Analytes with a positive %-Difference value (x-axis) are “Increased” in CVD cases compared to controls. Analytes with a negative %-Difference value (x-axis) are “Decreased” in CVD cases compared to controls.

FIG. 4: Examples of biomarker ratios that differ between CVD patients (case; squares) and healthy controls (control; circles).

FIG. 5: The ROC curves and Area Under the Curve (AUC) values for Cer(d18:1/18:0)/Total TAG ratio in total plasma (A), and LDL fraction (B), as well as LDL Cholesterol (C) measured from total plasma.

FIG. 6: A schematic diagram of a system according to some embodiments of the invention. In particular, this figure illustrates various hardware, software, and other resources that may be used in implementations of computer system 106 according to disclosed systems and methods. In embodiments as shown, computer system 106 may include one or more processors 110 coupled to random access memory operating under control of or in conjunction with an operating system. The processor(s) 110 in embodiments may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processor(s) 110 may communicate with data store 112, such as a database stored on a hard drive or drive array, to access or store program instructions other data.

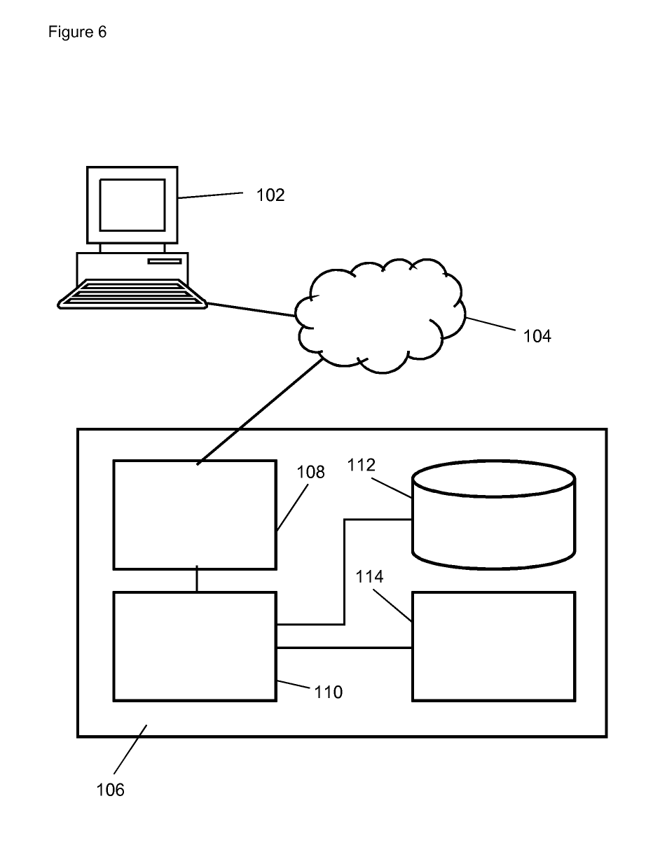

Processor(s) 110 may further communicate via a network interface 108, which in turn may communicate via the one or more networks 104, such as the Internet or other public or private networks, such that a query or other request may be received from client 102, or other device or service. Additionally, processor(s) 110 may utilize network interface 108 to send information, instructions, workflows query partial workflows, or other data to a user via the one or more networks 104. Network interface 104 may include or be communicatively coupled to one or more servers. Client 102 may be, e.g., a personal computer coupled to the internet.

Processor(s) 110 may, in general, be programmed or configured to execute control logic and control operations to implement methods disclosed herein. Processors 110 may be further communicatively coupled (i.e., coupled by way of a communication channel) to co-processors 114. Co-processors 114 can be dedicated hardware and/or firmware components configured to execute the methods disclosed herein. Thus, the methods disclosed herein can be executed by processor 110 and/or co-processors 114.

Other configurations of computer system 106, associated network connections, and other hardware, software, and service resources are possible.

### DETAILED DESCRIPTION OF THE INVENTION

The present invention is based on the finding that certain (non-protein) molecules are decreased and certain molecules are increased in non-HDL particles of patients having CVD, most likely due to alterations in the particle quality. Lipid ratios derived from these molecules were surprisingly superior biomarkers of CVD. According to the invention, (not cholesterol related) bioactive ceramides and glucosylated/galactosylated/ganglioside derivatives of ceramides, as well as esterified cholesterol (not total cholesterol), were present in lower concentrations in LDL particles of patients with CVD. We provide evidence indicating that low levels of these molecules in LDL particles may be harmful and can be associated with the development of CVD. These changes may be masked if these markers or standard markers are measured from total plasma/serum, and it is therefore an important aspect of this invention that said ratios are determined from a non-HDL fraction of plasma/serum samples, which preferably includes the LDL fraction.

Cardiovascular disease is a highly heterogeneous disease. Therefore, in order for the diagnostic measurement to work on an individual level, the individual variation should be normalized. TAG molecules were identified to be expressed in higher concentrations in LDL particles of CVD patients. The invention provides that ceramide, ceramide derivatives, esterified cholesterol and lysophospholipid molecule levels may be used in combination with TAG molecule levels to calculate a concentration ratio. This ratio improves diagnostic power to separate CVD patients from controls. A change in the ratio therefore reflects the individual's risk of having or developing CVD.

An association of CE/TAG ratio with the structure and composition of both LDL and HDL has been suggested in regard to CVD (Deckelbaum et al.). However, the decreased ratio was often interpreted to indicate increased incidence of the small, dense LDL particle type, suggested to be harmful. Moreover, recent studies have demonstrated that CVD risk assessment based on LDL particle size is not improved from the traditional total/HDL cholesterol ratio-based risk assessment (Parish et al., 2012). According to the present invention, specific CE and TAG molecules and their ratios, not LDL size, contribute to the increased risk of CVD.

Methods, according to the invention, provide specific and sensitive tests that can be used to identify and predict CVD. In addition, the present invention can be used for CVD and atherosclerosis diagnosis when conventional CVD and atherosclerosis markers are negative. The present invention may advantageously be used for patients on statin treatment to assess whether they are at high risk to develop CVD complications.

Generally, the invention provides methods of detecting atherosclerosis or CVD, and/or one or more of its complications, comprising the steps of measuring the levels of markers from non-HDL, for example from LDL fractions from a test sample (plasma or serum sample) of the subject, calculating a ratio of the marker levels, and determining if the ratio of the non-HDL-based markers correlates with atherosclerosis or CVD, and/or one or more of its complications.

Due to both high sensitivity and specificity of lipidomics, even the smallest sample amounts can be analyzed. Collecting information on a lipid biomarker (i.e., a lipid/lipid concentration ratio, as described and/or claimed herein) according to the methods of the invention from the subject's sample, and also from the control sample, can be performed via various chemical and high resolution analytical techniques. Particularly suitable analytical techniques include, but are not limited to, mass spectrometry and nuclear magnetic resonance spectroscopy. Indeed, any high resolution technique capable of resolving individual lipids or lipid classes and providing structural information of the same can be used to determine the lipid markers according to the invention from the subject's sample, and also from the control sample. For the purposes of the methods of the present invention the lipid concentration(s) or lipid/lipid concentration ratio(s) are thus preferably determined by using mass spectrometry. However, nuclear magnetic resonance spectroscopy, fluorescence spectroscopy or dual polarisation interferometry, high performance separation methods such as HPLC or UPLC, an immunoassay such as an ELISA and/or the use of a binding moiety capable of specifically binding the lipid analyte are also useful in this regard.

In the present invention, novel lipid biomarkers determined from a non-HDL fraction (e.g. the LDL fraction) of plasma/serum samples, have been identified. Surprisingly, the ratios of ceramide or ceramide derivatives (glucosylated/galactosylated/ganglioside), cholesteryl ester, lysophospholipid molecular species or the sum of any of these molecular species to triglyceride (TAG) molecular species or the sum of TAG molecular species, showed superior performance as CVD biomarkers compared to existing and conventionally used biomarkers, when measured from a non-HDL fraction, such as the LDL fraction.

In connection with the present invention, lipid ratios in the LDL fraction of total plasma have been determined. As LDL is the major class of the functionally related atherogenic non-HDL lipoprotein classes (VLDL, IDL and LDL), it is to be assumed that the ratios of the present invention may also be used in CVD risk assessment if measured from a non-HDL fraction other than the LDL fraction. Therefore, the diagnostic method according to the invention can also be used to measure said ratios from a non-HDL fraction of a blood plasma or serum sample, other than the LDL fraction.

### DEFINITIONS

As used herein, “CVD” is coronary vascular disease/cardiovascular disease and its general meaning in the art is to classify conditions that affect the heart, heart valves, blood, and vasculature of the body. “CAD” is coronary artery disease, “AMI” is acute myocardial infarction, “ACS” is acute coronary syndrome, “CAC” is coronary artery calcification, “RCT” is reverse cholesterol transport, “LDL” is low density lipoprotein, “HDL” is high density lipoprotein, “LDL-C” is low density lipoprotein cholesterol, “HDL-C” is high density lipoprotein cholesterol, “ApoA” is Apolipoprotein A, “ApoB” is Apolipoprotein B, “ApoC” is apolipoprotein C, “MS” is mass spectrometry, “HPLC” is high performance liquid chromatography, and “UPLC” is ultra performance liquid chromatography.

As used herein, “ceramide” refers to any ceramide-based molecule, including glucosylceramides, galactosylceramides and gangliosides (oligosaccharide-linked ceramides).

As used herein, “a subject” includes all mammals, including without limitation humans, but also non-human primates, dogs, cats, horses, sheep, goats, cows, rabbits, pigs and rodents. It will be appreciated that a particularly preferred subject according to the invention is a human subject.

A “sample” is defined as a biological sample obtained from a subject or a group or population of subjects.

A “non-HDL sample” is a blood serum or blood plasma sample, or a sample obtained from blood serum or blood plasma, e.g., a lipoprotein containing fraction obtained as described herein, which is substantially reduced in, essentially free of, or completely free of HDL particles. “Substantially reduced” in HDL particles means that less than 70% (w/w), preferably less than 80% (w/w), and more preferably less than 90% (w/w) of the lipoprotein in the sample is HDL. “Essentially free” of HDL particles means that less than 95% (w/w), and preferably less than 98% (w/w) of the lipoprotein in the sample is HDL. “Completely free” of HDL particles means that less than 99% (w/w), and preferably less than 99.5% (w/w) of the lipoprotein in the sample is HDL. Thus, a non-HDL sample in accordance with the invention is advantageously blood serum or blood plasma from which HDL particles have been removed by the methods described herein to render it substantially reduced in, essentially free of, or completely free of HDL particles, as explained above (i.e., plasma or serum minus HDL). Also preferred is, however, an embodiment where the non-HDL sample to be analyzed is an LDL sample, obtained from blood serum or blood plasma. It can also advantageously be a VLDL sample or an IDL sample, or combinations of LDL, VLDL and IDL samples, obtained from blood serum or blood plasma. All these non-HDL samples may be obtained by preparing a non-HDL fraction, an LDL fraction, a VLDL fraction, or an IDL fraction, respectively, from blood serum or blood plasma by methods known in the art and/or described herein. Moreover, taking a blood sample of a patient in order to obtain blood serum or blood plasma is part of normal clinical practice. The blood sample can be taken in connection with, e.g., measuring the cholesterol levels in the patients. The collected blood sample can be prepared and serum or plasma can be separated with techniques well known to a person skilled in the art. Vena blood samples can be collected from patients using a needle and a BD Vacutainer® Plastic Tubes or Vacutainer® Plus Plastic Tubes (BD Vacutainer® SST™ Tubes contain spray-coated silia and a polymer gel for serum separation). Serum can be separated by centrifugation at 1300 RCF for 10 min at room temperature and stored in small plastic tubes at −80° C.

A “non-HDL fraction” is defined as the fraction of total plasma/serum excluding HDL. It can comprise LDL, VLDL and/or IDL, or combinations thereof; wherein LDL is typically the major constituent. It is functionally distinct from the HDL-containing fraction. The non-HDL fraction can be prepared using any technique known in the art, including centrifugation and/or precipitation, for example by sequential differential micro-ultracentrifugation using potassium bromide solution as described in St∪hlman et al., or by precipitating as described in Burstein et al. Alternatively, the non-HDL fraction can be prepared by fast performance liquid chromatography (FPLC) as described for example in Wiesner et al. or by gel filtration methods, as described for example in Dallinga-Thie et al.

An “LDL fraction” is defined as the fraction of total plasma/serum including LDL and excluding HDL. In addition to LDL, it can comprise VLDL and/or IDL; wherein LDL is typically the major constituent. It is functionally distinct from the HDL-containing fraction. The LDL fraction can be prepared using any technique known in the art, including ultracentrifugation and/or precipitation, for example by sequential differential micro-ultracentrifugation using potassium bromide solution as described in St∪hlman et al. or by precipitating as described in Burstein et al.

A “VLDL fraction” is defined as the fraction of total plasma/serum including VLDL and excluding HDL. In addition to VLDL, it can comprise LDL and/or IDL; wherein VLDL is typically the major constituent. It is functionally distinct from the HDL-containing fraction. The VLDL fraction can be prepared using any technique known in the art, including ultracentrifugation and/or precipitation, for example by sequential differential micro-ultracentrifugation using potassium bromide solution as described in Stah∪man et al. or by precipitating as described in Burstein et al.

An “IDL fraction” is defined as the fraction of total plasma/serum including IDL and excluding HDL. In addition to IDL, it can comprise LDL and/or VLDL; wherein IDL is typically the major constituent. It is functionally distinct from the HDL-containing fraction. The IDL fraction can be prepared using any technique known in the art, including ultracentrifugation and/or precipitation, for example by sequential differential micro-ultracentrifugation using potassium bromide solution as described in Stah∪man et al. or by precipitating as described in Burstein et al.

An “HDL-containing fraction” is defined as the fraction of total plasma/serum including HDL particles. It typically excludes LDL, VLDL and IDL particles. It is functionally distinct from the non-HDL, LDL, VLDL and IDL fractions.

A “non-HDL sample” according to the invention will comprise, preferably consist essentially of, more preferably consist of or will correspond to material from the “non-HDL fraction” of total plasma/serum.

Likewise, an “LDL sample”, a “VLDL sample”, or an “IDL sample” in accordance with the invention will comprise, preferably consist essentially of, more preferably consist of or will correspond to material from the “LDL fraction”, the “VLDL fraction”, or the “IDL fraction” respectively, of the “non-HDL fraction” of total plasma/serum.

The term “control”, as used herein, may be a control sample. Alternatively, it may also be a control value. In case it is a control value, it will be appreciated that it may have already been determined, calculated or extrapolated prior to initiating the methods of the invention. Alternatively, the control value may be determined, calculated or extrapolated after determination of said lipid/lipid concentration ratio(s), in accordance with the methods of the present invention. Thus, it will be appreciated that a suitable control value in accordance with the present invention may well be one that is taken from the literature.

A “control” as used herein, i.e., a control value or a control sample, is typically representative of a group of subjects or a population of subjects. In this context, “representative” means that the lipid/lipid concentration(s) reflected by said control value to which a comparison is made in the context of the present invention correspond(s) to the average concentration value(s) of said lipid/lipid concentration ratio(s) in corresponding individual samples from the subjects of said group or population. Likewise, in the case of a control sample “representative” means that the lipid/lipid concentration(s) in said control sample to which a comparison is made in the context of the present invention correspond(s) to the average concentration(s) of said lipid/lipid concentration ratio(s) in corresponding individual samples from the subjects of said group or population. Preferably, the concentrations of all lipid/lipid concentration ratios in said control sample correspond to the average concentrations of said lipid/lipid concentration ratios in corresponding individual samples from the subjects of said group or population. An individual with such values can be considered a “healthy individual” for the purposes of the invention. In a preferred embodiment, a control sample from a group of subjects or a control sample from a population of subjects in the sense of the present invention is obtained by mixing equal amounts of samples directly obtained or taken from the subjects of said group or population, or by mixing equal amounts of fractions, constituents or reaction products (e.g., enzymatic reaction products or precipitates) thereof.

In the context of the present invention, a control sample can be from a healthy individual, a generalized population of healthy individuals, a CAD patient that has remained free of any major CVD complications, or a group of CAD patients that have remained free of any major CVD complications.

In the context of the present invention, the reference to a control sample from the same subject or from a(nother) subject may mean that the control sample has been directly obtained from said subject. Alternatively, however, it may also mean that it has been obtained as the result of a physical or chemical treatment of a sample directly obtained or taken from said subject, such as centrifugation, fractionation, enzymatic digestion, precipitation, and the like. The same applies to any reference herein to a control sample from a group of subjects or from a population of subjects.

A control sample can be particularly suitably compared to the subject's sample if it has been obtained from the same type of biological tissue or source in the same, or essentially the same, manner. For example, if the subject's sample is a non-HDL sample, an LDL sample, a VLDL sample or an IDL sample as defined herein, a corresponding control sample will likewise be a non-HDL sample, an LDL sample, a VLDL sample or an IDL sample, respectively. It will be appreciated that such a corresponding control sample would include a non-HDL sample, an LDL sample, a VLDL sample or an IDL sample that is obtained by mixing the respective non-HDL samples, LDL samples, VLDL samples or IDL samples from a group or population of subjects.

It will be appreciated that a useful control value for the purposes of the present invention is preferably one that has been, or is, obtained using any one of the suitable control samples described herein.

The term “lipid” as used herein is defined as a hydrophobic or an amphiphilic small molecule.

For the purposes of the present invention, lipids are named according to the following nomenclature: Cer is ceramide, Glc/GalCer is glucosyl- and galactosylceramides, GM is monosialogangliosides, GM3 is monosialodihexosylganglioside, CE is cholesteryl ester, LPE is lysophosphatidylethanolamine, TAG is triacylglycerol.

The nomenclature X:Y indicates, X number of total carbon atoms in the fatty acid(s) portions of the molecule, and Y the total number of double bonds in the fatty acid portion(s) of the molecule.

The nomenclature A/B/C indicates, for a molecule of TAG, A, B and C types of fatty acid moieties attached to the glycerol backbone of the molecule.

The nomenclature (dE/F) (e.g. Cer(d18:0/20:0)) indicates, for a molecule of Cer, GlcCer and GM, E, the type of long-chain base with an amide-linked, F, fatty acid moiety.

For a molecule of GM, the following number (e.g. GM2 and GM3) characterizes the carbohydrate sequence.

In the context of the present invention, a “ceramide” refers to any one of the lipids of the family of ceramide lipids, which includes glucosylated (Glc) derivatives, galactosylated (Gal) derivatives and ganglioside derivatives of ceramide (such as GM3), which are modified from ceramides by respective glucosyl or galactosyl synthases, or in the case of GM3, by beta-galactosidase and ganglioside GM3 synthase.

The term “TAG brutto species” is defined as a species of TAG molecules that consists of one or more possible combinations of fatty acids (see Table 7 for example).

A “ceramide/TAG concentration ratio” is defined as the ratio of the concentration of at least one ceramide molecule to the concentration of at least one TAG molecule, i.e. concentration of ceramide(s) divided by the concentration of TAG(s). Alternatively, it is the ratio of the concentration of at least one ceramide molecule to the concentration of any TAG brutto species, or it is the ratio of the concentration of the sum of all ceramide molecules to the concentration of the sum of all TAG molecules.

A “CE/TAG concentration ratio” is defined as the ratio of the concentration of at least one CE molecule to the concentration of at least one TAG molecule, i.e. concentration of CE(s) divided by the concentration of TAG(s). Alternatively, it is the ratio of the concentration of at least one CE molecule to the concentration of any TAG brutto species, or it is the ratio of the concentration of the sum of all CE molecules to the concentration of the sum of all TAG molecules.

A “LPE/TAG concentration ratio” is defined as the ratio of the concentration of at least one LPE molecule to the concentration of at least one TAG molecule, i.e., concentration of LPE(s) divided by the concentration of TAG(s). Alternatively, it is the ratio of the concentration of at least one LPE molecule to the concentration of any TAG brutto species, or it is the ratio of the concentration of the sum of all LPE molecules to the concentration of the sum of all TAG molecules.

A “ceramide/CE concentration ratio” is defined as the ratio of the concentration of at least one ceramide molecule to the concentration of at least one CE molecule, i.e. concentration of ceramide(s) divided by the concentration of CE(s). Alternatively, it is the ratio of the concentration of the sum of all ceramide molecules to the concentration of the sum of all CE molecules.

A “decreased ratio” is defined as a negative percentage difference (% difference) in the value of a lipid/lipid concentration ratio between a subject's sample and a control, such that a negative percentage difference for a particular lipid/lipid ratio between a subject′ sample and a control indicates that the lipid/lipid ratio has a smaller numerical value in the subject compared to the control. For example, a difference for a particular ratio of −50% means that the value of the particular ratio is 50% lower in the subject compared to the control.

The following calculation is an example calculation based on the average concentration values from Table 6.

Lipid ratio: CE 16:0/TAG 50:1 total (16:0/16:0/18:1)

### Concentrations in Controls:

CE 16:0 308.7 pmol/μg
TAG 50:1 total (16:0/16:0/18:1) 20.3 pmol/μg
Lipid ratio in controls: 308.7 pmol/μg/20.3 pmol/μg=15.2

### Concentrations in Cases:

CE 16:0 298.4 pmol/μg
TAG 50:1 total (16:0/16:0/18:1) 29.2 pmol/μg
Lipid ratio in cases: 298.4 pmol/μg/29.2 pmol/μg=10.2

### Percentage Difference Between Cases and Controls:

(Case ratio−Control ratio)/Case ratio=% difference

(10.2−15.2)/10.2=−0.49→×100%→−49%

The ratio is 50% lower in cases compared to controls.

A decreased ratio in the context of the present invention corresponds to a % difference in lipid/lipid ratio between subject and control of at least −10%, preferably at least −20%, −30%, or −40%, more preferably at least −50%, or also −60%, −70%, −80%, or −90%, and in particularly preferred embodiments at least −100%, but also at least −120%, at least −140%, at least −160%, at least −180% or at least −200%.

An “increased ratio” is defined as a positive percentage difference (% difference) in the value of a lipid/lipid ratio between a subject and a control, such that a positive percentage difference for a particular lipid/lipid ratio between a subject and a control indicates that the lipid/lipid ratio has a higher numerical value in the subject compared to the control. For example, a % difference for a particular ratio of 50% means that the value of the particular ratio is 50% higher in the subject compared to the control.

An increased ratio in the context of the present invention corresponds to a % difference in lipid/lipid ratio between subject and control of at least 10%, preferably at least 20%, 30%, or 40%, more preferably at least 50%, or also 60%, 70%, 80%, or 90%, and in particularly preferred embodiments at least 100%, but also at least 120%, at least 140%, at least 160%, at least 180% or at least 200%.

The term “markedly differ” means that a comparison of lipid/lipid ratios between a subject and a control reveals more than a 15% difference in either a positive or a negative direction between the values. Accordingly, the term “does not markedly differ” means that a comparison of lipid/lipid ratios between a subject and a control reveals less than a 15% difference (i.e., an insignificant difference) in either a positive or a negative direction between the values.

The term “computer-implemented method” in the context of the present invention means a method which utilizes a machine or apparatus to achieve its objective.

The term “processor” means a device which is capable of interpreting and executing instructions. Specifically, a processor employs logic circuitry to receive input data and provide the appropriate output data. Processors can communicate with each other via a network.

The term “effectiveness of a treatment” is taken to mean the ability of a treatment to achieve the therapeutic purpose for which it is administered.

In the context of all aspects and embodiments of the invention described and/or claimed herein, a “statin” may be selected from, but not limited to, the group consisting of atorvastatin, cerivastatin, fluvastatin, fluvastatin XL, lovastatin, pitavastatin, pravastatin, rosuvastatin and simvastatin.

A “modulator” according to the invention may be a small molecule (<1500 dalton molecular weight, preferably <800 dalton molecular weight), an antibody, an antisense RNA, a small interfering RNA (siRNA), or a natural or modified lipid, preferably a lipid of any one of the lipid/lipid concentration ratios referred to in Tables 1 to 4.

A “lipid lowering drug” according to the invention is preferably an HMG-CoA reductase inhibitor, niacin (nicotinic acid), a cholesterol absorption inhibitor, a cholesteryl ester transfer protein (CETP) inhibitor, a bile acid sequestrant, a fibrate, a phytosterol or a PCSK9 inhibitor.

For the purposes of the present invention, a “cholesterol absorption inhibitor” is preferably ezetimibe or SCH-48461; a cholesteryl ester transfer protein (CETP) inhibitor is preferably evacetrapib, anacetrapib or dalcetrapib; a bile acid sequestrant is preferably colesevelam, cholestyramine or colestipol; a fibrate is preferably fenofibrate, gemfibrozil, clofibrate, or bezafibrate, and the PCSK9 inhibitor is selected from a PCSK9 specific antibody, an siRNA, and a peptidomimetic.

As used herein, the term “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies of the invention include monoclonal and polyclonal antibodies, whole antibodies, antibody fragments, and antibody sub-fragments that exhibit specific binding to a said lipid. Thus, suitable “antibodies” can be whole immunoglobulins of any class, e.g., IgG, IgM, IgA, IgD, IgE, chimeric antibodies or hybrid antibodies with dual or multiple antigen or epitope specificities, or fragments, e.g., F(ab′)₂, Fab′, Fab and the like, including hybrid fragments, and additionally includes any immunoglobulin or any natural, synthetic or genetically engineered protein that acts like an antibody by binding to a specific antigen to form a complex. The term “antibody” encompasses antigen-binding fragments of antibodies (e.g., single chain antibodies, Fab fragments, F(ab′)₂, a Fd fragment, a Fv fragment and dAb fragments) as well as complete antibodies. For example, Fab molecules can be expressed and assembled in a genetically transformed host like E. coli. A lambda vector system is available thus to express a population of Fab's with a potential diversity equal to or exceeding that of the subject generating the predecessor antibody. See Huse W D, et al., Science 1989, 246:1275-81. Such Fab's are included in the definition of “antibody.” The ability of a given molecule, including an antibody fragment or sub-fragment, to act like an antibody and specifically bind to a specific antigen can be determined by binding assays known in the art, for example, using the antigen of interest as the binding partner.

Antibodies against lipids in accordance with the present invention may be prepared by methods well known to those skilled in the art. For example, mice may be immunized with a lipid with adjuvant. Splenocytes are harvested as a pool from the mice that were administered 3 immunizations at 2-week intervals with test bleeds performed on alternate weeks for serum antibody titers. Splenocytes are prepared as 3 aliquots that are either used immediately in fusion experiments or stored in liquid nitrogen for use in future fusions.

Fusion experiments are then performed according to the procedure of Stewart & Fuller, J. Immunol. Methods 1989, 123:45-53. Supernatants from wells with growing hybrids are screened by enzyme-linked immunosorbent assay (ELISA) for monoclonal antibody (MAb) secretors on 96-well ELISA plates coated with the said lipid. ELISA positive cultures are cloned by limiting dilutions, typically resulting in hybridomas established from single colonies after 2 serial cloning experiments.

For the purpose of the present invention an “enzyme” is an enzyme suitable for detection in an ELISA. Such enzymes are known to those skilled in the art. Such enzyme may, for example, be acetylcholinesterase, horseradish peroxidase, or alkaline phosphatase.

Similarly, a “detectable label” in the sense described herein is one that is suitable for detection in an ELISA, EIA (enzyme immunoassay), or a RIA (radioimmunoassay). Again, those skilled in the art will be familiar with suitable detectable labels, which may be, for example, biotin or another hapten. Alternatively, it may be a fluorescent label such as FITC, TRITC, Texas Red, rhodamine, phycoerythrin (PE), APC, Cy-3, Cy-5, Cy-7, an Alexa Fluor, a DyLight Fluor, an ATTO Dye or any other suitable fluorescent label or variation thereof. In the case where biotin is the detectable label, streptavidin conjugated to an enzyme or detectable label may be used for its detection. The detectable label may also be a radio-isotope, such as ¹²⁵I, ¹³¹I or ³²P.

The terms “of the invention”, “in accordance with the invention”, or “according to the invention” as used herein are intended to refer to all aspects and embodiments of the invention described and/or claimed herein.

As used herein, the term “comprising” is to be construed as encompassing both “including” and “consisting of”, both meanings being specifically intended, and hence individually disclosed, embodiments in accordance with the present invention.

### EXAMPLES

### Example 1

### Materials and Methods

Serum samples were obtained from the Health2000 Survey, which included a thorough health examination and interview of the participants. Patients with coronary symptoms (angina pectoris or myocardial infarction) and healthy subjects were included.

Low-density lipoprotein (LDL) particles were fractionated from serum samples of 19 patients with coronary artery disease (non-lipid based diagnosis) and 10 healthy controls by sequential differential micro-ultracentrifugation using potassium bromide solution as described in Stah∪man et al. LDL particles were collected in one fraction at density d=1.063. Beckman Coulter Optima MAX-XP ultracentrifuge and TLA-120.2 rotor were used for lipoprotein isolation.

Total protein content of the LDL samples was determined using the Micro BCA™ Protein Assay Kit prior to lipid extraction. Lipid extraction followed by established/validated platforms providing quantitative molecular lipidomics analyses of LDL samples were performed as previously described in Deckelbaum et al. Obtained lipid data was normalized to sample protein concentration.

For quantification of ceramides (Cer), triacylglycerols (TAG), cholesteryl esters (CE) and lysophospholipids, lipids were extracted using a modified Folch lipid extraction performed on a Hamilton Microlab Star robot, as described in Jung et al. Samples were spiked with known amounts of non-endogeneous synthetic internal standards. After lipid extraction, samples were reconstituted in chloroform:methanol (1:2, v/v) and a synthetic external standard was post-extract spiked to the extracts. The extracts were stored at −20° C. prior to MS analysis.

Gangliosides were extracted according to Fong and colleagues (2009) with minor modifications. Samples were spiked with known amounts of non-endogeneous synthetic internal standard. After lipid extraction, samples were reconstituted in chloroform:methanol (1:2, v/v) and stored at −20° C. prior to MS analysis.

In Shotgun Lipidomics, lipid extracts were analyzed using a hybrid triple quadrupole/linear ion trap mass spectrometer (QTRAP, ABSciex) equipped with a robotic nanoflow ion source (NanoMate HD, Advion Biosciences) according to Stah∪man and colleagues (2008). Molecular lipids were analyzed in both positive and negative modes using Multiple Precursor Ion Scanning (MPIS) based methods according to Ekroos et al. (2002, 2003). Triacylglycerols (TAG) were analyzed using Precursor Ion Scanning (PIS) and Neutral Loss scanning (NL) based methods. The molecular lipid species were identified and quantified in semi-absolute or absolute amounts as described in Ejsing et al. (2006).

Sphingolipids were analyzed on a hybrid triple quadrupole/linear ion trap mass spectrometer (QTRAP) equipped with an ultra high pressure liquid chromatography (UHPLC) system (CTC HTC PAL autosampler and Rheos Allegro pump) using multiple reaction monitoring (MRM)-based method in negative ion mode based on the description by Sullards et al. (2007).

Masses and counts of detected peaks by mass spectrometry were converted into a list of corresponding lipid names and concentrations. Calibration lines were generated to determine the dynamic quantification range for each lipid class monitored, e.g., the quantification limits. Internal standards were used for quantifying endogenous lipid species. Calibration lines were used to determine the quantification limits of the method.

For each platform, a stringent cutoff was applied for separating background noise from actual lipid peaks. Each sample was controlled and only accepted when fulfilling the acceptance criteria. Masses and counts of detected peaks were converted into a list of corresponding lipid names. Lipids were normalized to their respective internal standard and sample volume to retrieve their concentrations.

Unpaired Student t-tests were conducted to log transformed lipid concentrations due to approximate log normality of the data. In case of lipid/lipid ratios, t-tests were applied to log transformed ratios which were approximately log normal. ROC curves, odds ratios (OR) and all the related descriptive statistics were derived from logistic regression model which was fitted on log transformed lipid concentrations and lipid/lipid ratios. Normality assumptions of the used methods were tested with the Shapiro-Wilk test. All the analyses were performed using SAS 9.3 software. Reported differences between the group means were calculated from the original scale.

### Example 2

### Results

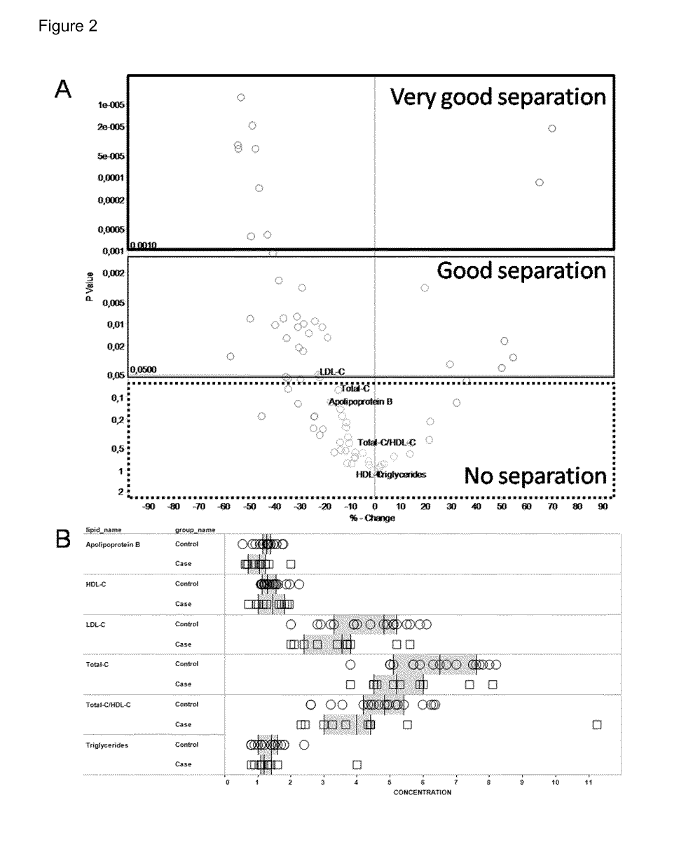

Standard lipid parameters are routinely measured from plasma/serum samples of individuals to assess the risk of CVD of individuals. As part of the Health2000 Survey, a thorough health examination including standard lipid parameters was performed to obtain the overall health status of the general population. In general, high levels of apolipoprotein B, total cholesterol (total-C), LDL cholesterol (LDL-C), and triglycerides, and on the other hand low levels of HDL cholesterol (HDL-C), are regarded as risk factors. An elevated ratio of total vs. HDL cholesterol is also used to evaluate the risk of CVD. As discussed earlier however, these parameters fail to identify a large proportion of CVD patients. We compared the results from standard lipid tests of patients with verified CVD to those of control individuals. There were no significant differences in any of the standard lipid tests between cases and controls, as shown in FIG. 2A. All of these currently used CVD markers were found in the bottom (dashed) box of the Volcano Plot (FIG. 2A), meaning that these markers did not differ between cases and controls. This demonstrates the poor performance of present methods in separating CVD patients based on laboratory tests. The concentrations of the standard tests are illustrated in FIG. 2B. In fact, the CVD patients of this cohort had more beneficial lipid results than controls, including lower Apolipoprotein B, total-C and LDL-C. Of note, lower LDL-C cannot be explained by lipid-lowering medication as there were no statin users among these participants.

Routinely measured standard lipid tests are measured at the level of total plasma/serum, including the specific HDL-C and LDL-C tests. As the function of a specific lipid may differ depending on the lipoprotein particle it is associated with, we decided to isolate LDL fractions of the samples for further analysis. These fractions were analyzed comprehensively for their lipid content. The lipid data consists of concentrations of molecular lipids (i.e. lipid class and fatty acid moieties identified) from several lipid classes as well as lipid class concentrations, obtained by summing up the molecular lipid concentrations of that lipid class.

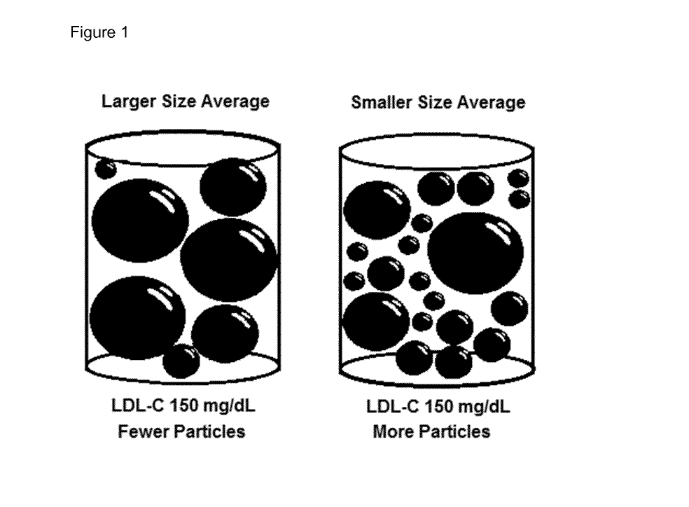

In clinical use, lipid concentrations are normalized against the total sample volume, and the concentration unit is, for example, given as mmol/l or mg/dl. The LDL fraction consists of a heterogeneous population of particles that differ in their physical properties, including size, density and charge, as well as in their amount of lipid molecules. This means that, with a given lipid concentration the number of particles may vary substantially between individuals (see FIG. 1) which could result in a marked bias in risk estimation. To even out the differences in particle numbers in the sample, suggested to be an informative marker of CVD risk, we normalized the lipid data against the total protein concentration of the sample.

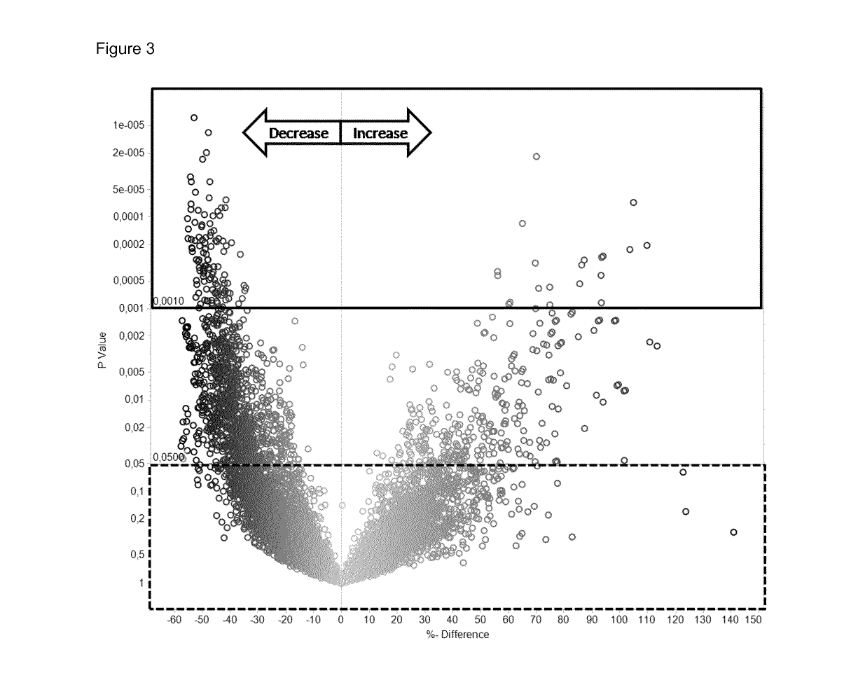

From these lipid data, a number of lipid ratios together with the statistical significance of each ratio between cases and controls, were calculated using concentrations of molecular lipid species, and/or total lipid class concentrations (derived as described above). Combining concentrations of individual lipids into distinct lipid/lipid ratios markedly improves the predictive value to separate cases from controls. In FIG. 3, the molecular lipid and lipid class concentrations, as well as calculated lipid/lipid ratios are presented as a Volcano plot. This plot illustrates the difference of each analyte (circles) between CVD cases and controls with color intensity, and the statistical significance thereof. Circles in the bottom box (dashed) represent analytes that do not differ between CVD patients and controls. The majority of quantified lipids and calculated ratios, as well as standard lipid tests, fall into the bottom box (FIG. 3). On the other hand, a number of lipid ratios differed highly significantly (p<0.001) between the groups (FIG. 3, upper box) and separated CVD cases from controls. The location of the circles along the x-axis indicates decreased or increased level of that analyte in CVD patients compared to controls. Table 1 summarizes the highly significant (p<0.001) lipid ratios. For each lipid ratio, percentage difference (% Difference) and odds ratios per standard deviation (OR per SD) with 95% confidence interval (95% CI) together with their P values, are presented. The concentrations of the molecular lipids used in these ratios are presented in Table 6.

The vast majority of the identified ratios of Table 1 are composed of ceramide (Cer), ceramide derivative (Glc/GalCer/GM3 ganglioside) or cholesteryl ester (CE) lipids to triacylglycerol (TAG) lipids. In addition, ratios of lysophosphatidylethanolamine (LPE) lipids to TAG lipids differed significantly between CVD patients and controls. Importantly, ratios composed of molecular lipid species as well as lipid class sums are able to separate the two groups significantly. These ratios therefore serve as superior CVD biomarkers compared to the currently used conventional lipid tests.

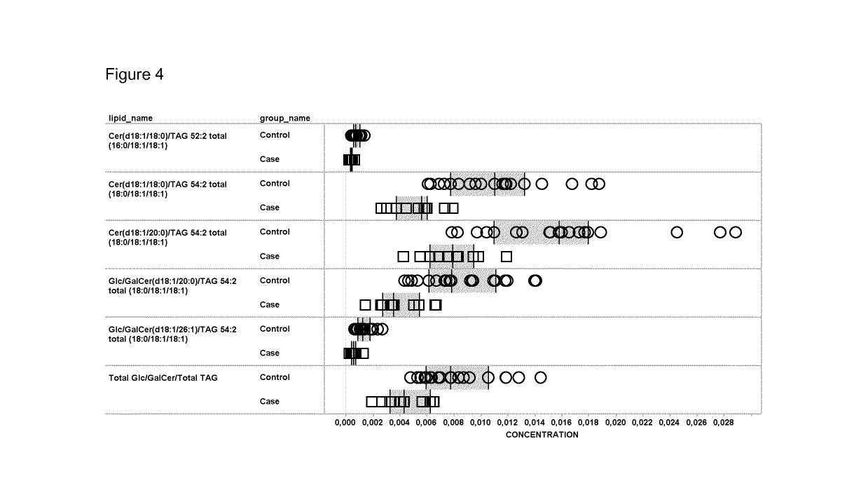

The current invention relates to the use of ratios of ceramide, ceramide derivatives or cholesterol ester to triglycerides in a non-HDL fraction as CVD biomarkers. Said triglyceride molecular species are presented as molecule sums (for example TAG 52:2 total) that can consist of one or more possible combinations of fatty acids according to Table 7. Said biomarkers may be composed of concentrations of said molecular lipids and/or lipid class sums. Some examples of the biomarker ratios are presented in FIG. 4.

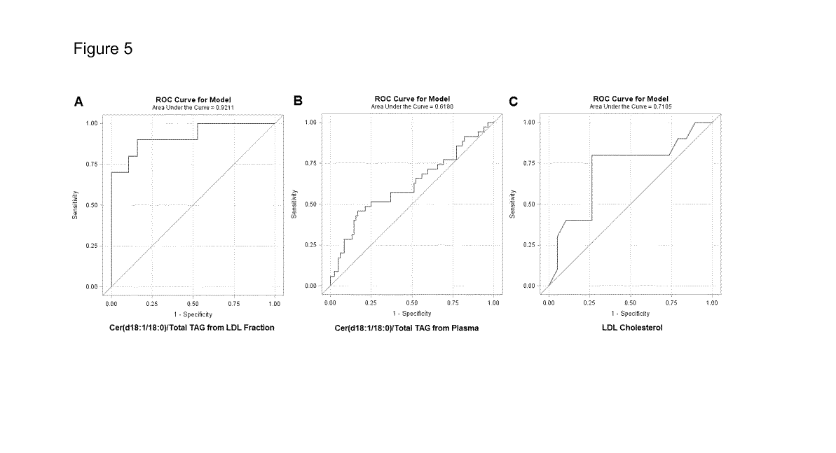

An important aspect of this invention is that said lipid ratios are determined from non-HDL fractions, such as the LDL fraction, which yield superior discrimination of CVD patients and controls compared to the same lipid ratios from total blood/plasma/serum (FIG. 5, Table 5). The ROC curves and Area Under Curve (AUC) values are shown for the Cer(d18:1/18:0)/Total TAG ratio in the LDL fraction (FIG. 5A) and in total plasma (FIG. 5A). The ROC curve for LDL Cholesterol (FIG. 5C) is shown for comparison. Additional AUC values of plasma/LDL lipid ratios are listed in Table 5. This demonstrates the diagnostic improvement according to this invention, for determining lipid ratios from a specific fraction instead of a total blood/plasma sample.

TABLE 1
List of statistically highly significantly affected ceramide/TAG ratios between CVD and healthy
subjects in alphabetical order. Abbreviations: Cer, ceramide; TAG, triacylglycerol; CI, confidence interval;
OR, odds ratio; SD, standard deviation.

| Lipid | | % | Difference | OR per SD | |
|---|---|---|---|---|---|
| ratio | Lipid Names | Difference | P Value | (95% CI) | OR P Value |
| Cer/TAG | Cer(d18:1/16:0)/TAG 50:1 total (16:0/16:0/18:1) | −48.752421 | 0.000166098 | 0.05 (0.01-0.47) | 0.008516792 |
| | Cer(d18:1/16:0)/TAG 50:2 total | −47.20322653 | 0.00015799 | 0.05 (0-0.49) | 0.010679812 |
| | (14:0/18:1/18:1)(16:0/16:0/18:2)(16:0/16:1/18:1) | | | | |
| | Cer(d18:1/16:0)/TAG 50:3 total | −42.08091904 | 0.000523248 | 0.11 (0.02-0.63) | 0.013132572 |
| | (14:0/18:1/18:2)(16:0/16:1/18:2)(16:1/16:1/18:1) | | | | |
| | Cer(d18:1/16:0)/TAG 52:1 total (16:0/18:0/18:1) | −44.27468227 | 0.001009493 | 0.07 (0.01-0.69) | 0.022208178 |
| | Cer(d18:1/16:0)/TAG 52:2 total (16:0/18:1/18:1) | −42.57694689 | 0.000180974 | 0.06 (0.01-0.60) | 0.016289902 |
| | Cer(d18:1/16:0)/TAG 54:2 total (18:0/18:1/18:1) | −47.54491485 | 6.10151E−05 | 0.05 (0-0.51) | 0.01218665 |
| | Cer(d18:1/16:0)/TAG 54:3 total | −44.37414538 | 0.000345047 | 0.11 (0.02-0.60) | 0.011104454 |
| | (18:0/18:1/18:2)(18:1/18:1/18:1) | | | | |
| | Cer(d18:1/16:0)/Total TAG | −41.79547104 | 7.79439E−05 | 0.07 (0.01-0.53) | 0.009663408 |
| | Cer(d18:1/18:0)/TAG 50:1 total (16:0/16:0/18:1) | −54.06151621 | 7.11956E−05 | 0.03 (0-0.57) | 0.019753252 |
| | Cer(d18:1/18:0)/TAG 50:2 total | −52.67281363 | 0.00011323 | 0.08 (0.01-0.50) | 0.007100191 |
| | (14:0/18:1/18:1)(16:0/16:0/18:2)(16:0/16:1/18:1) | | | | |
| | Cer(d18:1/18:0)/TAG 50:3 total | −48.08116181 | 0.000290459 | 0.13 (0.03-0.57) | 0.00702318 |
| | (14:0/18:1/18:2)(16:0/16:1/18:2)(16:1/16:1/18:1) | | | | |
| | Cer(d18:1/18:0)/TAG 52:1 total (16:0/18:0/18:1) | −50.25309842 | 0.000126003 | 0.01 (0-0.58) | 0.026040316 |
| | Cer(d18:1/18:0)/TAG 52:2 total (16:0/18:1/18:1) | −48.52580266 | 1.95365E−05 | 0.03 (0-0.47) | 0.012222699 |
| | Cer(d18:1/18:0)/TAG 52:3 total | −43.73805728 | 9.54262E−05 | 0.10 (0.02-0.57) | 0.010067355 |
| | (16:0/18:1/18:2)(16:1/18:1/18:1) | | | | |
| | Cer(d18:1/18:0)/TAG 54:2 total (18:0/18:1/18:1) | −52.97910407 | 8.11792E−06 | 0.01 (0-0.53) | 0.021925869 |
| | Cer(d18:1/18:0)/TAG 54:3 total | −50.13681679 | 0.00012045 | 0.04 (0-0.56) | 0.016915397 |
| | (18:0/18:1/18:2)(18:1/18:1/18:1) | | | | |
| | Cer(d18:1/18:0)/TAG 56:6 total (18:1/18:1/20:4) | −43.19362704 | 7.83745E−05 | 0.06 (0-0.62) | 0.018705794 |
| | Cer(d18:1/18:0)/Total TAG | −47.82528536 | 1.18026E−05 | 0.05 (0.01-0.47) | 0.008563296 |
| | Cer(d18:1/20:0)/TAG 50:1 total (16:0/16:0/18:1) | −51.07967684 | 0.00035324 | 0.08 (0.01-0.54) | 0.009378047 |
| | Cer(d18:1/20:0)/TAG 50:2 total | −49.60083442 | 0.00036983 | 0.12 (0.03-0.54) | 0.005562702 |
| | (14:0/18:1/18:1)(16:0/16:0/18:2)(16:0/16:1/18:1) | | | | |
| | Cer(d18:1/20:0)/TAG 50:3 total | −44.71114124 | 0.00067035 | 0.18 (0.05-0.62) | 0.006637052 |
| | (14:0/18:1/18:2)(16:0/16:1/18:2)(16:1/16:1/18:1) | | | | |
| | Cer(d18:1/20:0)/TAG 52:1 total (16:0/18:0/18:1) | −46.71394776 | 0.000805399 | 0.06 (0-0.63) | 0.020057495 |
| | Cer(d18:1/20:0)/TAG 52:2 total (16:0/18:1/18:1) | −45.18464346 | 0.000179325 | 0.10 (0.02-0.56) | 0.00830346 |
| | Cer(d18:1/20:0)/TAG 52:3 total | −40.08612841 | 0.001098672 | 0.22 (0.07-0.67) | 0.007752838 |
| | (16:0/18:1/18:2)(16:1/18:1/18:1) | | | | |
| | Cer(d18:1/20:0)/TAG 54:2 total (18:0/18:1/18:1) | −49.92700599 | 2.31097E−05 | 0.04 (0-0.46) | 0.009716428 |
| | Cer(d18:1/20:0)/TAG 54:3 total | −46.90022755 | 0.000145218 | 0.10 (0.02-0.57) | 0.009189241 |
| | (18:0/18:1/18:2)(18:1/18:1/18:1) | | | | |
| | Cer(d18:1/20:0)/TAG 56:6 total (18:1/18:1/20:4) | −39.50635952 | 0.000188787 | 0.12 (0.03-0.58) | 0.007750974 |
| | Cer(d18:1/20:0)/Total TAG | −44.438656 | 8.54937E−05 | 0.13 (0.03-0.52) | 0.004224931 |
| | Cer(d18:1/22:0)/TAG 50:1 total (16:0/16:0/18:1) | −53.58109129 | 0.00023659 | 0.08 (0.01-0.57) | 0.012601799 |
| | Cer(d18:1/22:0)/TAG 50:2 total | −52.17786565 | 0.000288951 | 0.11 (0.02-0.58) | 0.009345276 |
| | (14:0/18:1/18:1)(16:0/16:0/18:2)(16:0/16:1/18:1) | | | | |
| | Cer(d18:1/22:0)/TAG 50:3 total | −47.53819431 | 0.000479329 | 0.16 (0.04-0.63) | 0.009057205 |
| | (14:0/18:1/18:2)(16:0/16:1/18:2)(16:1/16:1/18:1) | | | | |
| | Cer(d18:1/22:0)/TAG 52:1 total (16:0/18:0/18:1) | −49.15063459 | 0.000338764 | 0.08 (0.01-0.70) | 0.022509865 |
| | Cer(d18:1/22:0)/TAG 52:2 total (16:0/18:1/18:1) | −47.98748522 | 0.000289811 | 0.08 (0.01-0.62) | 0.015578464 |
| | Cer(d18:1/22:0)/TAG 52:3 total | −43.14966957 | 0.000710325 | 0.17 (0.04-0.63) | 0.008486871 |
| | (16:0/18:1/18:2)(16:1/18:1/18:1) | | | | |
| | Cer(d18:1/22:0)/TAG 54:2 total (18:0/18:1/18:1) | −52.48735928 | 5.29829E−05 | 0.04 (0-0.58) | 0.019368016 |
| | Cer(d18:1/22:0)/TAG 54:3 total | −49.61534734 | 0.000441818 | 0.06 (0.01-0.65) | 0.020586911 |
| | (18:0/18:1/18:2)(18:1/18:1/18:1) | | | | |
| | Cer(d18:1/22:0)/TAG 56:6 total (18:1/18:1/20:4) | −42.59954567 | 0.000480576 | 0.12 (0.03-0.60) | 0.00931006 |
| | Cer(d18:1/22:0)/Total TAG | −47.2796419 | 0.000116605 | 0.08 (0.01-0.56) | 0.01060759 |
| | Cer(d18:1/24:0)/TAG 54:2 total (18:0/18:1/18:1) | −48.31909669 | 0.000813514 | 0.11 (0.02-0.62) | 0.01193207 |
| | Cer(d18:1/24:1)/TAG 50:1 total (16:0/160/18:1) | −48.52009701 | 0.000608121 | 0.08 (0.01-0.56) | 0.011370363 |
| | Cer(d18:1/24:1)/TAG 50:2 total | −46.96387948 | 0.000459534 | 0.08 (0.01-0.61) | 0.015225147 |
| | (14:0/18:1/18:1)(16:0/16:0/18:2)(16:0/16:1/18:1) | | | | |
| | Cer(d18:1/24:1)/TAG 50:3 total | −41.8183507 | 0.000628271 | 0.11 (0.02-0.63) | 0.013117455 |
| | (14:0/18:1/18:2)(16:0/16:1/18:2)(16:1/16:1/18:1) | | | | |
| | Cer(d18:1/24:1)/TAG 52:2 total (16:0/18:1/18:1) | −42.31662723 | 0.000235581 | 0.04 (0-0.59) | 0.018240847 |
| | Cer(d18:1/24:1)/TAG 52:3 total | −36.95135073 | 0.000878437 | 0.15 (0.04-0.63) | 0.009213082 |
| | (16:0/18:1/18:2)(16:1/18:1/18:1) | | | | |
| | Cer(d18:1/24:1)/TAG 54:2 total (18:0/18:1/18:1) | −47.3071168 | 4.07174E−05 | 0.04 (0-0.46) | 0.010207713 |
| | Cer(d18:1/24:1)/TAG 54:3 total | −44.12197307 | 0.000170274 | 0.05 (0.01-0.55) | 0.01355825 |
| | (18:0/18:1/18:2)(18:1/18:1/18:1) | | | | |
| | Cer(d18:1/24:1)/TAG 56:6 total (18:1/18:1/20:4) | −36.34124752 | 0.000253129 | 0.08 (0.01-0.56) | 0.010326536 |
| | Cer(d18:1/24:1)/Total TAG | −41.53160866 | 6.42362E−05 | 0.03 (0-0.50) | 0.014523864 |
| | Total Cer/TAG 54:2 total (18:0/18:1/18:1) | −93.47556594 | 0.000272754 | 13.30 (1.81-97.83) | 0.011018646 |
| | Total Cer/TAG 54:3 total | −82.44712561 | 0.001138831 | 7.68 (1.48-39.95) | 0.015361807 |
| | (18:0/18:1/18:2)(18:1/18:1/18:1) | | | | |
| | Total d18:1 ceramides/TAG 54:3 total | −82.92609499 | 0.001083691 | 7.85 (1.48-41.48) | 0.015297638 |
| | (18:0/18:1/18:2)(18:1/18:1/18:1) | | | | |
| | Total Cer/TAG 56:6 total (18:1/18:1/20:4) | −60.14742672 | 0.000884847 | 6.89 (1.59-29.91) | 0.009911384 |
| | Total d18:1 ceramides/TAG 56:6 total | −60.56785381 | 0.000848238 | 6.98 (1.60-30.50) | 0.009824386 |
| | (18:1/18:1/20:4) | | | | |
| | Total Cer/Total TAG | −42.64872769 | 0.000603575 | 0.11 (0.02-0.65) | 0.014348107 |
| | Total d18:1 ceramides/Total TAG | −74.82179733 | 0.000578675 | 9.07 (1.55-52.91) | 0.01427796 |
| Glc/Gal | Glc/GalCer(d18:1/16:0)/TAG 54:2 total | −42.6878551 | 0.000951748 | 0.14 (0.03-0.64) | 0.011141958 |
| Cer/ | (18:0/18:1/18:1) | | | | |
| TAG | Glc/GalCer(d18:1/18:0)/TAG 50:1 total | −52.10795906 | 0.000488565 | 0.10 (0.02-0.59) | 0.011045001 |
| | (16:0/16:0/18:1) | | | | |
| | Glc/GalCer(d18:1/18:0)/TAG 50:2 total | −50.66020121 | 0.000882456 | 0.13 (0.03-0.67) | 0.014860639 |
| | (14:0/18:1/18:1)(16:0/16:0/18:2)(16:0/16:1/18:1) | | | | |
| | Glc/GalCer(d18:1/18:0)/TAG 52:2 total | −46.33683652 | 0.000616647 | 0.15 (0.04-0.64) | 0.009971645 |
| | (16:0/18:1/18:1) | | | | |
| | Glc/GalCer(d18:1/18:0)/TAG 54:2 total | −50.97951681 | 0.000204656 | 0.14 (0.04-0.57) | 0.005637086 |
| | (18:0/18:1/18:1) | | | | |
| | Glc/GalCer(d18:1/18:0)/TAG 54:3 total | −48.01635983 | 0.00081905 | 0.17 (0.04-0.66) | 0.010933335 |
| | (18:0/18:1/18:2)(18:1/18:1/18:1) | | | | |
| | Glc/GalCer(d18:1/18:0)/TAG 56:6 total | −40.77790744 | 0.000666425 | 0.16 (0.04-0.64) | 0.009366469 |
| | (18:1/18:1/20:4) | | | | |
| | Glc/GalCer(d18:1/18:0)/Total TAG | −45.60652936 | 0.000478938 | 0.12 (0.02-0.62) | 0.011970804 |
| | Glc/GalCer(d18:1/20:0)/TAG 50:1 total | −55.04078833 | 0.000133758 | 0.04 (0-0.61) | 0.021020952 |
| | (16:0/16:0/18:1) | | | | |
| | Glc/GalCer(d18:1/20:0)/TAG 50:2 total | −53.68168877 | 0.000218475 | 0.07 (0.07-0.60) | 0.015645537 |
| | (14:0/18:1/18:1)(16:0/16:0/18:2)(16:0/16:1/18:1) | | | | |
| | Glc/GalCer(d18:1/18:0)/TAG 50:3 total | −49.18791734 | 0.00080153 | 0.13 (0.03-0.65) | 0.012986495 |
| | (14:0/18:1/18:2)(16:0/16:1/18:2)(16:1/16:1/18:1) | | | | |
| | Glc/GalCer(d18:1/20:0)/TAG 52:1 total | −51.98420651 | 0.000540009 | 0.04 (0-0.53) | 0.014394168 |
| | (16:0/18:0/18:1) | | | | |
| | Glc/GalCer(d18:1/20:0)/TAG 52:2 total | −49.62307977 | 0.000243073 | 0.11 (0.02-0.57) | 0.008866958 |
| | (16:0/18:1/18:1) | | | | |
| | Glc/GalCer(d18:1/20:0)/TAG 54:2 total | −53.98145001 | 4.07269E−05 | 0.07 (0.01-0.49) | 0.007918652 |
| | (18:0/18:1/18:1) | | | | |
| | Glc/GalCer(d18:1/20:0)/TAG 54:3 total | −51.19975185 | 0.000290981 | 0.14 (0.03-0.63) | 0.010700818 |
| | (18:0/18:1/18:2)(18:1/18:1/18:1) | | | | |
| | Glc/GalCer(d18:1/18:0)/TAG 56:6 total | −44.4045703 | 0.000705583 | 0.16 (0.04-0.65) | 0.01099809 |
| | (18:1/18:1/20:4) | | | | |
| | Glc/GalCer(d18:1/20:0)/Total TAG | −48.93749541 | 0.000222238 | 0.09 (0.01-0.59) | 0.011912562 |
| | Glc/GalCer(d18:1/22:0)/TAG 50:1 total | −55.32685281 | 0.000102912 | 0.04 (0-0.66) | 0.024037939 |
| | (16:0/16:0/18:1) | | | | |
| | Glc/GalCer(d18:1/22:0)/TAG 50:2 total | −53.97640087 | 0.000173957 | 0.06 (0.01-0.66) | 0.020716648 |
| | (14:0/18:1/18:1)(16:0/16:0/18:2)(16:0/16:1/18:1) | | | | |
| | Glc/GalCer(d18:1/22:0)/TAG 50:3 total | −49.5112222 | 0.000407293 | 0.10 (0.02-0.63) | 0.014271167 |
| | (14:0/18:1/18:2)(16:0/16:1/18:2)(16:1/16:1/18:1) | | | | |
| | Glc/GalCer(d18:1/22:0)/TAG 52:2 total | −49.9436158 | 0.00017632 | 0.09 (0.01-0.61) | 0.013149102 |
| | (16:0/18:1/18:1) | | | | |
| | Glc/GalCer(d18:1/22:0)/TAG 52:3 total | −45.28774481 | 0.000680072 | 0.14 (0.03-0.65) | 0.011950505 |
| | (16:0/18:1/18:2)(16:1/18:1/18:1) | | | | |
| | Glc/GalCer(d18:1/22:0)/TAG 54:2 total | −54.2742548 | 3.60126E−05 | 0.04 (0-0.59) | 0.018971304 |
| | (18:0/18:1/18:1) | | | | |
| | Glc/GalCer(d18:1/22:0)/TAG 54:3 total | −51.51025591 | 0.000302081 | 0.10 (0.02-0.60) | 0.012505469 |
| | (18:0/18:1/18:2)(18:1/18:1/18:1) | | | | |
| | Glc/GalCer(d18:1/22:0)/TAG 56:6 total | −44.75831044 | 0.000184079 | 0.07 (0.01-0.54) | 0.010969077 |
| | (18:1/18:1/20:4) | | | | |
| | Glc/GalCer(d18:1/22:0)/Total TAG | −49.26239365 | 9.3085E−05 | 0.05 (0-0.60) | 0.018336691 |
| | Glc/GalCer(d18:1/24:0)/TAG 50:1 total | −55.15665582 | 0.000170067 | 0.06 (0.01-0.61) | 0.016749449 |
| | (16:0/16:0/18:1) | | | | |
| | Glc/GalCer(d18:1/24:0)/TAG 50:2 total | −53.8010589 | 0.000214304 | 0.07 (0.01-0.67) | 0.021531098 |
| | (14:0/18:1/18:1)(16:0/16:0/18:2)(16:0/16:1/18:1) | | | | |
| | Glc/GalCer(d18:1/24:0)/TAG 50:3 total | −49.31886867 | 0.000499666 | 0.09 (0.01-0.63) | 0.014794401 |
| | (14:0/18:1/18:2)(16:0/16:1/18:2)(16:1/16:1/18:1) | | | | |
| | Glc/GalCer(d18:1/24:0)/TAG 52:2 total | −49.75290961 | 0.000353382 | 0.11 (0.02-0.63) | 0.012684883 |
| | (16:0/18:1/18:1) | | | | |
| | Glc/GalCer(d18:1/24:0)/TAG 54:2 total | −54.1000476 | 8.16695E−05 | 0.07 (0.01-0.57) | 0.012883588 |
| | (18:0/18:1/18:1) | | | | |
| | Glc/GalCer(d18:1/24:0)/TAG 54:3 total | −51.32551835 | 0.000671333 | 0.13 (0.03-0.65) | 0.012216797 |
| | (18:0/18:1/18:2)(18:1/18:1/18:1) | | | | |
| | Glc/GalCer(d18:1/24:0)/TAG 56:6 total | −44.54784913 | 0.000561678 | 0.11 (0.02-0.63) | 0.013873219 |
| | (18:1/18:1/20:4) | | | | |
| | Glc/GalCer(d18:1/24:0)/Total TAG | −49.06909212 | 0.000188996 | 0.06 (0.01-0.62) | 0.017457288 |
| | Glc/GalCer(d18:1/24:1)/TAG 50:1 total | −52.61216995 | 0.000178771 | 0.04 (0-0.48) | 0.011857847 |
| | (16:0/16:0/18:1) | | | | |
| | Glc/GalCer(d18:1/24:1)/TAG 50:2 total | −51.1796542 | 0.000164445 | 0.03 (0-0.56) | 0.019609454 |
| | (14:0/18:1/18:1)(16:0/16:0/18:2)(16:0/16:1/18:1) | | | | |
| | Glc/GalCer(d18:1/24:1)/TAG 50:3 total | −46.44313705 | 0.000418266 | 0.06 (0.01-0.54) | 0.012615067 |
| | (14:0/18:1/18:2)(16:0/16:1/18:2)(16:1/16:1/18:1) | | | | |
| | Glc/GalCer(d18:1/24:1)/TAG 52:2 total | −46.9018062 | 0.000207267 | 0.09 (0.01-0.57) | 0.010481396 |
| | (16:0/18:1/18:1) | | | | |
| | Glc/GalCer(d18:1/24:1)/TAG 54:2 total | −51.49560801 | 8.34753E−05 | 0.08 (0.01-0.51) | 0.007372168 |
| | (18:0/18:1/18:1) | | | | |
| | Glc/GalCer(d18:1/24:1)/TAG 54:3 total | −48.56364737 | 0.000252843 | 0.12 (0.02-0.62) | 0.011331076 |
| | (18:0/18:1/18:2)(18:1/18:1/18:1) | | | | |
| | Glc/GalCer(d18:1/24:1)/TAG 56:6 total | −41.40140193 | 0.000203892 | 0.13 (0.03-0.56) | 0.006282738 |
| | (18:1/18:1/20:4) | | | | |
| | Glc/GalCer(d18:1/24:1)/Total TAG | −46.17918777 | 9.597E−05 | 0.05 (0-0.51) | 0.011981999 |
| | Total Glc/GalCer/TAG 50:1 total (16:0/16:0/18:1) | −109.7500847 | 0.000201984 | 20.57 (1.81-234) | 0.014779054 |
| | Total Glc/GalCer/TAG 50:2 total | −103.5954724 | 0.00022412 | 20.49 (1.55-270) | 0.021813495 |
| | (14:0/18:1/18:1)(16:0/16:0/18:2)(16:0/16:1/18:1) | | | | |
| | Total Glc/GalCer/TAG 50:3 total | −85.58968578 | 0.000530685 | 12.76 (1.58-103) | 0.016963849 |
| | (14:0/18:1/18:2)(16:0/16:1/18:2)(16:1/16:1/18:1) | | | | |
| | Total Glc/GalCer/TAG 52:1 total (16:0/18:0/18:1) | −93.3539266 | 0.000854638 | 8.82 (1.62-47.97) | 0.011792095 |
| | Total Glc/GalCer/TAG 52:2 total (16:0/18:1/18:1) | −87.19283378 | 0.000292318 | 9.58 (1.67-55.05) | 0.01131096 |
| | Total Glc/GalCer/TAG 54:2 total (18:0/18:1/18:1) | −104.9216774 | 6.86079E−05 | 14.64 (1.86-116) | 0.01088292 |
| | Total Glc/GalCer/TAG 54:3 total | −93.240789 | 0.000429377 | 7.84 (1.57-39.16) | 0.012094311 |
| | (18:0/18:1/18:2)(18:1/18:1/18:1) | | | | |
| | Total Glc/GalCer/TAG 56:6 total (18:1/18:1/20:4) | −69.62182875 | 0.000315152 | 8.95 (1.69-47.53) | 0.010057693 |
| | Total Glc/GalCer/Total TAG | −45.85214211 | 0.000139129 | 0.05 (0-0.58) | 0.016330674 |
| GM3/ | GM3-d18:1/16:0/TAG 54:2 total (18:0/18:1/18:1) | −44.62727355 | 0.000866369 | 0.13 (0.03-0.63) | 0.011202794 |
| TAG | GM3-d18:1/18:0/TAG 54:2 total (18:0/18:1/18:1) | −47.58019159 | 0.000370199 | 0.11 (0.02-0.56) | 0.007463096 |
| | GM3-d18:1/18:0/TAG 54:3 total | −44.4115545 | 0.001124577 | 0.18 (0.05-0.66) | 0.009625029 |
| | (18:0/18:1/18:2)(18:1/18:1/18:1) | | | | |
| | GM3-d18:1/18:0/Total TAG | −41.83461435 | 0.00102188 | 0.16 (0.04-0.66) | 0.011033472 |
| | GM3-d18:1/20:0/TAG 54:2 total (18:0/18:1/18:1) | −48.73514992 | 0.000337253 | 0.10 (0.02-0.56) | 0.008436377 |
| | GM3-d18:1/22:0/TAG 54:2 total (18:0/18:1/18:1) | −47.59852265 | 0.000353485 | 0.11 (0.02-0.56) | 0.008022188 |
| | GM3-d18:1/24:0/TAG 54:2 total (18:0/18:1/18:1) | −49.58171839 | 0.000387986 | 0.13 (0.03-0.58) | 0.007417309 |
| | GM3-d18:1/24:1/TAG 54:2 total (18:0/18:1/18:1) | −45.47552978 | 0.000663984 | 0.13 (0.03-0.63) | 0.0113227 |
| | Total GM3/TAG 54:2 total (18:0/18:1/18:1) | −86.29374823 | 0.000330584 | 9.42 (1.78-49.87) | 0.008326932 |
| | Total GM3/TAG 54:3 total | −75.67468387 | 0.001110228 | 5.06 (1.49-17.20) | 0.009334768 |
| | (18:0/18:1/18:2)(18:1/18:1/18:1) | | | | |
| | Total GM3/Total TAG | −40.43777651 | 0.001080782 | 0.16 (0.04-0.66) | 0.011128257 |

TABLE 2
List of statistically highly significantly affected CE/TAG ratios between CVD and healthy subjects
in alphabetical order. Abbreviations: CE, cholesteryl ester; TAG, triacylglycerol; CI, confidence interval;
OR, odds ratio; SD, standard deviation.

| Lipid | | % | Difference | OR per SD | OR |
|---|---|---|---|---|---|
| ratio | Lipid Names | Difference | P Value | (95% CI) | P Value |
| CE/TAG | CE 16:0/TAG 50:1 total (16:0/16:0/18:1) | −41.77988901 | 0.000635295 | 0.08 (0.01-0.50) | 0.007055675 |
| | CE 16:0/TAG 50:2 total | −40.01991761 | 0.000672668 | 0.10 (0.02-0.57) | 0.009271536 |
| | (14:0/18:1/18:1)(16:0/16:0/18:2)(16:0/16:1/18:1) | | | | |
| | CE 16:0/TAG 52:2 total (16:0/18:1/18:1) | −34.76420564 | 0.00063125 | 0.19 (0.05-0.74) | 0.015958213 |
| | CE 16:0/TotalTAG | −33.87640544 | 0.001037532 | 0.16 (0.04-0.65) | 0.011035706 |
| | CE 18:1/TAG 50:1 total (16:0/16:0/18:1) | −42.24238441 | 0.000668698 | 0.09 (0.02-0.53) | 0.007960104 |
| | CE 18:1/TAG 50:2 total | −40.49639406 | 0.000770178 | 0.12 (0.02-0.60) | 0.009471792 |
| | (14:0/18:1/18:1)(16:0/16:0/18:2)(16:0/16:1/18:1) | | | | |
| | CE 18:1/TAG 54:2 total (18:0/18:1/18:1) | −40.88148743 | 0.000523992 | 0.09 (0.01-0.58) | 0.010910903 |
| | CE 18:1/Total TAG | −34.40168541 | 0.000590023 | 0.13 (0.03-0.65) | 0.013031498 |
| | CE 18:2/TAG 54:2 total (18:0/18:1/18:1) | −42.01672171 | 0.000423203 | 0.12 (0.02-0.59) | 0.008907275 |
| | CE 18:2/Total TAG | −35.6613493 | 0.000769184 | 0.15 (0.04-0.65) | 0.01080422 |
| | CE 22:6/TAG 50:1 total (16:0/16:0/18:1) | −52.34329499 | 0.000745707 | 0.10 (0.01-0.66) | 0.017451128 |
| | CE 22:6/TAG 50:2 total | −50.90265125 | 0.000381581 | 0.08 (0.01-0.69) | 0.021781292 |
| | (14:0/18:1/18:1)(16:0/16:0/18:2)(16:0/16:1/18:1) | | | | |
| | CE 22:6/TAG 50:3 total | −46.13925946 | 0.000393586 | 0.11 (0.02-0.63) | 0.013081488 |
| | (14:0/18:1/18:2)(16:0/16:1/18:2)(16:1/16:1/18:1) | | | | |
| | CE 22:6/TAG 52:2 total (16:0/18:1/18:1) | −46.60053106 | 0.000863207 | 0.10 (0.01-0.65) | 0.016802733 |
| | CE 22:6/TAG 52:3 total | −41.6337113 | 0.000797672 | 0.12 (0.02-0.63) | 0.012520056 |
| | (16:0/18:1/18:2)(16:1/18:1/18:1) | | | | |
| | CE 22:6/TAG 54:2 total (18:0/18:1/18:1) | −51.22039776 | 0.000974666 | 0.14 (0.03-0.68) | 0.015423985 |
| | CE 22:6/Total TAG | −45.87381255 | 0.000344987 | 0.07 (0.01-0.62) | 0.01632663 |
| | Total CE/TAG 50:1 total (16:0/16:0/18:1) | −74.85142488 | 0.000904531 | 10.74 (1.75-65.78) | 0.010270424 |
| | Total CE/TAG 50:2 total | −69.72082992 | 0.000991382 | 7.54 (1.63-34.85) | 0.009701666 |
| | (14:0/18:1/18:1)(16:0/16:0/18:2)(16:0/16:1/18:1) | | | | |
| | Total CE/TAG 52:2 total (16:0/18:1/18:1) | −56.04729677 | 0.000390543 | 6.27 (1.44-27.28) | 0.014458112 |
| | Total CE/TAG 54:2 total (18:0/18:1/18:1) | −70.82637813 | 0.000596554 | 8.81 (1.62-47.86) | 0.011702092 |

TABLE 3
List of statistically highly significantly affected LPE/TAG ratios between CVD and healthy subjects
in alphabetical order. Abbreviations: LPE, lysophosphatidylethanolamine; TAG, triacylglycerol; CI,
confidence interval; OR, odds ratio; SD, standard deviation.

| Lipid | | % | Difference P | OR per SD | OR P |
|---|---|---|---|---|---|
| ratio | Lipid Names | Difference | Value | (95% CI) | Value |
| LPE/ | LPE 16:0/TAG 50:2 total | −43.19159064 | 0.000996081 | 0.11 (0.02−0.67) | 0.016217869 |
| TAG | (14:0/18:1/18:1)(16:0/16:0/18:2)(16:0/16:1/18:1) | | | | |
| | LPE 18:0/TAG 50:1 total (16:0/16:0/18:1) | −46.44305484 | 0.00091767 | 0.11 (0.02−0.62) | 0.012432671 |
| | LPE 18:0/TAG 50:2 total | −44.82404913 | 0.000829839 | 0.12 (0.02−0.66) | 0.015423019 |
| | (14:0/18:1/18:1)(16:0/16:0/18:2)(16:0/16:1/18:1) | | | | |
| | LPE 18:0/TAG 54:2 total (18:0/18:1/18:1) | −45.18113493 | 0.00053068 | 0.12 (0.02−0.58) | 0.008888866 |

TABLE 4
List of statistically highly significantly affected ceramide/CE ratios between CVD and healthy
subjects in alphabetical order. Abbreviations: Cer, ceramide; CE, cholesteryl ester; CI, confidence interval;
OR, odds ratio; SD, standard deviation.

| Lipid | | % | Difference | OR per SD | |
|---|---|---|---|---|---|
| ratio | Lipid Names | Difference | P Value | (95% CI) | OR P Value |
| Cer/CE | Cer(d18:1/18:0)/CE 22:0 | −70.05576628 | 2.15393E−05 | 20.0 (2.09-193) | 0.009371028 |
| | Cer(d18:1/20:0)/CE 22:0 | −56.11901368 | 0.000431534 | 10.35 (1.51-70.90) | 0.017297373 |
| | Cer(d18:1/22:0)/CE 22:0 | −65.01134108 | 0.000116348 | 13.17 (1.93-89.62) | 0.008427653 |
| | Cer(d18:1/18:0)/CE 22:0 | −70.05576628 | 2.15393E−05 | 20.0 (2.09-193) | 0.009371028 |

TABLE 5
Comparison of Area Under Curve (AUC) values with 95% confidence interval
(95% CI) of select lipid ratios between plasma and LDL fraction,
demonstrating markedly better discrimination in LDL fraction.

| | AUC (95% CI) | |
|---|---|---|
| Lipid Names | Total plasma | LDL fraction |
| Cer(d18:1/18:0)/Total TAG | 0.62 (0.50-0.74) | 0.92 (0.81-1.00) |
| Cer(d18:1/20:0)/Total TAG | 0.60 (0.48-0.73) | 0.89 (0.77-1.00) |
| Cer(d18:1/24:1)/Total TAG | 0.62 (0.51-0.74) | 0.93 (0.82-1.00) |
| Glc/GalCer(d18:1/22:0)/Total TAG | 0.50 (0.38-0.63) | 0.88 (0.75-1.00) |
| Glc/GalCer(d18:1/24:1)/Total TAG | 0.54 (0.42-0.67) | 0.92 (0.81-1.00) |

TABLE 6
Average lipid concentrations (pmol/μg of total protein) of the
molecular lipids used to calculate ratios of Table 1.

| | Concentration | |
|---|---|---|
| Lipid Name | CVD patient | Control |
| CE 16:0 | 298.436 | 308.734 |
| CE 18:1 | 493.614 | 520.839 |
| CE 18:2 | 1218.582 | 1328.557 |
| CE 22:0 | 1.373 | 1.082 |
| CE 22:6 | 45.213 | 50.363 |
| Cer(d18:1/16:0) | 0.092 | 0.104 |
| Cer(d18:1/18:0) | 0.056 | 0.056 |
| Cer(d18:1/20:0) | 0.078 | 0.077 |
| Cer(d18:1/22:0) | 0.637 | 0.728 |
| Cer(d18:1/24:0) | 3.211 | 3.485 |
| Cer(d18:1/24:1) | 1.321 | 1.473 |
| Glc/GalCer(d18:1/16:0) | 0.333 | 0.412 |
| Glc/GalCer(d18:1/18:0) | 0.032 | 0.046 |
| Glc/GalCer(d18:1/20:0) | 0.029 | 0.041 |
| Glc/GalCer(d18:1/22:0) | 0.314 | 0.445 |
| Glc/GalCer(d18:1/24:0) | 0.493 | 0.692 |
| Glc/GalCer(d18:1/24:1) | 0.351 | 0.507 |
| GM3-d18:1/16:0 | 3.955 | 4.764 |
| GM3-d18:1/18:0 | 2.470 | 2.849 |
| GM3-d18:1/20:0 | 0.719 | 0.776 |
| GM3-d18:1/22:0 | 1.310 | 1.485 |
| GM3-d18:1/24:0 | 0.896 | 1.056 |
| GM3-d18:1/24:1 | 2.134 | 2.545 |
| LPE 16:0 | 0.473 | 0.492 |
| LPE 18:0 | 0.651 | 0.674 |
| TAG 50:1 total (16:0/16:0/18:1) | 29.188 | 20.286 |
| TAG 50:2 total (14:0/18:1/18:1)(16:0/16:0/18:2)(16:0/16:1/18:1) | 40.117 | 31.148 |
| TAG 50:3 total (14:0/18:1/18:2)(16:0/16:1/18:2)(16:1/16:1/18:1) | 14.563 | 12.220 |
| TAG 52:1 total (16:0/18:0/18:1) | 8.840 | 6.032 |
| TAG 52:2 total (16:0/18:1/18:1) | 106.587 | 83.909 |
| TAG 52:3 total (16:0/18:1/18:2)(16:1/18:1/18:1) | 81.169 | 67.655 |
| TAG 54:2 total (18:0/18:1/18:1) | 8.843 | 6.025 |
| TAG 54:3 total (18:0/18:1/18:2)(18:1/18:1/18:1) | 29.748 | 22.944 |
| TAG 56:6 total (18:1/18:1/20:4) | 2.218 | 1.773 |

TABLE 7
Possible fatty acid combinations per TAG brutto species.

| TAG brutto species | Possible fatty acid combinations |
|---|---|
| TAG 50:1 total | (16:0/16:0/18:1) |
| TAG 50:2 total | (14:0/18:1/18:1) |
| | (16:0/16:0/18:2) |
| | (16:0/16:1/18:1) |
| TAG 50:3 total | (14:0/18:1/18:2) |
| | (16:0/16:1/18:2) |
| | (16:1/16:1/18:1) |
| TAG 52:1 total | (16:0/18:0/18:1) |
| TAG 52:2 total | (16:0/18:1/18:1) |
| TAG 52:3 total | (16:0/18:1/18:2) |
| | (16:1/18:1/18:1) |
| TAG 54:2 total | (18:0/18:1/18:1) |
| TAG 54:3 total | (18:0/18:1/18:2) |
| | (18:1/18:1/18:1) |
| TAG 56:6 total | (18:1/18:1/20:4) |

### FURTHER EMBODIMENTS OF THE INVENTION

In view of the above, it will be appreciated that the present invention also encompasses the embodiments set forth in the numbered items of the following paragraphs.
1. A method for determining whether a subject is at risk to develop, or is suffering from atherosclerosis or cardiovascular disease (CVD) and/or one or more of its complications, said method comprising
   (a) determining in a non-HDL sample from said subject a ceramide/TAG concentration ratio, wherein a decreased ratio in said sample, when compared to a control, is indicative of said subject suffering from or having an increased risk of developing atherosclerosis or CVD and/or one or more of its complications;
   (b) determining in a non-HDL sample from said subject a CE/TAG concentration ratio, wherein a decreased ratio in said sample, when compared to a control, is indicative of said subject suffering from or having an increased risk of developing atherosclerosis or CVD and/or one or more of its complications;
   (c) determining in a non-HDL sample from said subject a LPE/TAG concentration ratio, wherein a decreased ratio in said sample, when compared to a control, is indicative of said subject suffering from or having an increased risk of developing atherosclerosis or CVD and/or one or more of its complications; or
   (d) determining in a non-HDL sample from said subject a ceramide/CE concentration ratio, wherein an decreased ratio in said sample, when compared to a control, is indicative of said subject suffering from or having an increased risk of developing atherosclerosis or CVD and/or one or more of its complications.
2. A method for evaluating the effectiveness of a treatment of atherosclerosis or CVD and/or one or more of its complications in a subject, comprising
   (a) determining in a non-HDL sample from said subject a ceramide/TAG concentration ratio, wherein a decreased ratio in said sample, when compared to a control, is indicative of the effectiveness of said treatment;
   (b) determining in a non-HDL sample from said subject a CE/TAG concentration ratio, wherein a decreased ratio in said sample, when compared to a control, is indicative of the effectiveness of said treatment;
   (c) determining in a non-HDL sample from said subject a LPE/TAG concentration ratio, wherein a decreased ratio in said sample, when compared to a control, is indicative of the effectiveness of said treatment; or
   (d) determining in a non-HDL sample from said subject a ceramide/CE concentration ratio, wherein an decreased ratio in said sample, when compared to a control, is indicative of the effectiveness of said treatment.
3. A method of choosing an appropriate treatment of atherosclerosis or CVD and/or one or more of its complications in a subject, comprising
   (a) determining in a non-HDL sample from said subject a ceramide/TAG concentration ratio, wherein a decreased ratio in said sample, when compared to a control, is indicative of said subject being in need of treatment or a change in, or supplementation of, an already administered treatment;
   (b) determining in a non-HDL sample from said subject a CE/TAG concentration ratio, wherein a decreased ratio in said sample, when compared to a control, is indicative of said subject being in need of treatment or a change in, or supplementation of, an already administered treatment;
   (c) determining in a non-HDL sample from said subject a LPE/TAG concentration ratio, wherein a decreased ratio in said sample, when compared to a control, is indicative of said subject being in need of treatment or a change in, or supplementation of, an already administered treatment; or
   (d) determining in a non-HDL sample from said subject a ceramide/CE concentration ratio, wherein an decreased ratio in said sample, when compared to a control, is indicative of said subject being in need of treatment or a change in, or supplementation of, an already administered treatment.
4. The method of any one of items 1 to 3, wherein
   (a) the ceramide/TAG concentration ratio whose decrease is compared to the control is selected from any of the ceramide/TAG concentration ratios referred to in Table 1;
   (b) the CE/TAG concentration ratio whose decrease is compared to the control is selected from any of the CE/TAG concentration ratios referred to in Table 2;
   (c) the LPE/TAG concentration ratio whose decrease is compared to the control is selected from any of the LPE/TAG concentration ratios referred to in Table 3; or
   (d) the ceramide/CE concentration ratio whose decrease is compared to the control is selected from any of the ceramide/CE concentration ratios referred to in Table 4.
5. The method of any one of items 1 to 4, wherein determining the lipid/lipid concentration ratio(s) is done using an assay.
6. The method of items 2 or 3, wherein said treatment is a lipid modifying treatment.
7. The method of any one of the preceding items, wherein
   (a) the ceramide/TAG concentration ratio whose decrease is compared to the control is selected from:
      Glc/GalCer(d18:1/22:0)/TAG 50:1 total (16:0/16:0/18:1);
      Cer(d18:1/22:0)/TAG 50:1 total (16:0/16:0/18:1); or
      GM3-d18:1/24:0/TAG 54:2 total (18:0/18:1/18:1);
   (b) the CE/TAG concentration ratio whose decrease is compared to the control is selected from:
      CE 22:6/TAG 50:1 total (16:0/16:0/18:1);
      CE 16:0/TAG 50:1 total (16:0/16:0/18:1); or
      CE 22:6/TAG 50:2 total (14:0/18:1/18:1)(16:0/16:0/18:2)(16:0/16:1/18:1);
   (c) the LPE/TAG concentration ratio whose decrease is compared to the control is selected from:
      LPE 18:0/TAG 50:1 total (16:0/16:0/18:1);
      LPE 18:0/TAG 50:2 total (14:0/18:1/18:1)(16:0/16:0/18:2)(16:0/16:1/18:1); or
      LPE 16:0/TAG 50:2 total (14:0/18:1/18:1)(16:0/16:0/18:2)(16:0/16:1/18:1); or
   (d) the ceramide/CE concentration ratio whose increase is compared to the control is selected from:
      Cer(d18:1/18:0)/CE 22:0;
      Cer(d18:1/20:0)/CE 22:0; or
      Cer(d18:1/22:0)/CE 22:0.
8. The method of any one of the preceding items, comprising determining at least 2, at least 3, at least 4, at least 5, at least 6, at least 7, or at least 8 of the lipid/lipid concentration ratios referred to therein, or combinations thereof.
9. The method of any one of the preceding items, wherein
   (a) said CVD is characterized by coronary artery disease, peripheral artery disease, a stroke and/or CVD death; and/or
   (b) said CVD is atherosclerosis-induced; and/or
   (c) said subject has atherosclerosis; or
   (d) said subject does not have atherosclerosis.
10. The method of any one of the preceding items, wherein
   (a) the method further comprises determining the serum level of total cholesterol, low-density lipoprotein cholesterol (LDL-C), high-density lipoprotein cholesterol (HDL-C), Apolipoprotein B (ApoB) and/or Apolipoprotein C-III (ApoC-III) in a sample from said subject; and/or
   (b) the subject does not have elevated serum levels of one or more of total cholesterol, low-density lipoprotein cholesterol (LDL-C), Apolipoprotein C-III (ApoC-III) or Apolipoprotein B (ApoB), or a decreased serum level of HDL-cholesterol (HDL-C).
11. The method of any one of the preceding items, wherein said subject
   (a) is being or has been treated with a statin, another lipid lowering drug, and/or a modulator of lipid/lipid concentration ratios; or
   (b) has not yet undergone statin therapy, therapy with another lipid lowering drug, and/or therapy with a modulator of lipid/lipid concentration ratios.
12. The method of any one of the preceding items, wherein the non-HDL sample is
   a LDL sample,
   a very-low density lipoprotein (VLDL) sample, or
   an intermediate-density lipoprotein (IDL) sample, or combinations thereof.
13. The method of any one of the preceding items, wherein the non-HDL sample is an LDL sample.
14. The method of any one of the preceding items, wherein the lipid/lipid concentration ratio is determined by using mass spectrometry, nuclear magnetic resonance spectroscopy, fluorescence spectroscopy or dual polarisation interferometry, a high performance separation method such as HPLC, UHPLC or UPLC, an immunoassay such as an ELISA and/or an assay with a binding moiety capable of specifically binding the analyte.
15. The method of any one of the preceding items, wherein the method is for:
   (a) determining a risk of said patient to develop CVD;
   (b) determining early warning signs of CVD in said patient;
   (c) determining or predicting the occurrence of atherosclerosis in a patient; and/or
   (d) predicting and/or diagnosing CVD and/or CVD complications including predicting and/or diagnosing myocardial infarction (MI), angina pectoris, transient ischemic attack (TIA) and stroke, or predicting death.
16. A drug capable of modulating a lipid/lipid concentration ratio according to any one of items 1 to 4 or 7, for use in treating or preventing atherosclerosis or CVD and/or one or more of its complications, wherein the drug is administered such that said lipid/lipid concentration ratio in a sample from said subject does not markedly differ when compared to a control, and wherein the drug is a statin, another lipid lowering drug, or a modulator of lipid/lipid concentration ratios.
17. A method of treating or preventing atherosclerosis or CVD and/or one or more of its complications, in a subject in need thereof, comprising administering a therapeutically effective dose of a drug capable of modulating a lipid/lipid concentration ratio according to any one of items 1 to 4 or 7, wherein the drug is administered such that said lipid/lipid concentration ratio does not markedly differ when compared to a control, and wherein the drug is a statin, another lipid lowering drug, or a modulator of lipid/lipid concentration ratios.
18. A kit for predicting or detecting atherosclerosis or CVD and/or one or more of its complications in a subject, or for performing the methods or uses according to any one of the preceding or following items, wherein the kit comprises:
   (a) one or more lipid standards chosen from the lipids in any one of the lipid/lipid concentration ratios referred to in items 1 to 4 or 7; and/or (a) calibration line control(s); and/or positive and/or negative controls; and optionally one or more of the following:
   (b) one or more control markers, such as a lipid or lipids, e.g., a lipid of any one of the lipid/lipid concentration ratios referred to in items 1 to 4 or 7, or a protein;
   (c) internal and/or external standards;
   (d) an agent, optionally an antibody, capable of binding any one of the lipids in any one of the lipid/lipid concentration ratios referred to in items 1 to 4 or 7; and
   (e) (a) reagent(s) for performing said methods or uses.
19. Use of a kit as defined in item 18 for predicting or detecting atherosclerosis or CVD and/or one or more of its complications, wherein the lipid/lipid concentration ratio in a sample from a subject is optionally determined by using mass spectrometry.
20. A kit for predicting or detecting atherosclerosis or CVD and/or one or more of its complications in a subject, or for performing the methods or uses according to any one of the preceding items, wherein the kit comprises:
   (a) (an) antibody(ies) capable of binding any one of the lipids in any one of the lipid/lipid concentration ratios referred to in items 1 to 4 or 7 conjugated to an enzyme or a detectable label; or any one of the lipid(s) in any one of the lipid/lipid concentration ratios referred to in items 1 to 4 or 7 conjugated to an enzyme or a detectable label; and optionally one or more of the following:
   (b) a substrate specific for said enzyme;
   (c) an assay plate coated with (a) secondary antibody(ies) capable of binding any of the antibodies of (a);
   (d) (a) standard(s) and/or (a) calibration line standard(s);
   (e) a stop solution; and
   (f) necessary buffers and/or reagents required to perform the assay.
21. The kit of item 20, wherein the antibody conjugated to an enzyme or a detectable label is capable of binding to any one of the lipids in the lipid/lipid concentration ratios referred to in item 7; and/or wherein the lipid conjugated to an enzyme or a detectable label is any one of the lipids in the lipid/lipid concentration ratios referred to in item 7.
22. An antibody against any one of the lipids in any one of the lipid/lipid concentration ratios referred to in items 1 to 4 or 7 for use in
   a) predicting a risk of a subject to develop, or to suffer from atherosclerosis or CVD and/or one or more of its complications; or
   b) preventing or treating atherosclerosis or CVD and/or one or more of its complications in a subject.
23. The method, drug, kit, use, or antibody of any one of the preceding items, wherein the subject is at risk to develop or has suffered from one or more CVD complications such as acute myocardial infarction and/or is at risk of cardiovascular death.
24. The method, drug, kit, use, or antibody of any one of the preceding items, wherein the subject has suffered from a cardiovascular disease.
25. A statin, another lipid lowering drug, or a modulator of lipid/lipid concentration ratios for use for preventing or treating atherosclerosis or CVD and/or one or more of its complications in a subject, wherein
   a) said subject would be identified as being at risk to develop, or as suffering from atherosclerosis or CVD and/or one or more of its complications when applying any of the methods, drugs, kits, uses, or antibodies of any one of the preceding items;
   b) said subject has been identified as being at risk to develop, or as suffering from atherosclerosis or CVD and/or one or more of its complications by any of the methods, drugs, kits, uses, or antibodies of any one of the preceding items;
   c) said subject would be identified as not being at risk to develop, or as suffering from atherosclerosis or CVD and/or one or more of its complications when applying any of the methods, drugs, kits, uses, or antibodies of any one of the preceding items; and/or
   d) said subject has been identified as not being at risk to develop, or as suffering from atherosclerosis or CVD and/or one or more of its complications by any of the methods, drugs, kits, uses, or antibodies of any one of the preceding items.
26. The method of any one of items 11 to 15, 17, 23, or 24, the drug of any one of items 16, 23, or 24, or the statin, other lipid lowering drug, or modulator of lipid/lipid concentration ratios of item 25, wherein the lipid lowering drug is selected from an HMG-CoA reductase inhibitor other than a statin; niacin (nicotinic acid); a cholesterol absorption inhibitor; a cholesteryl ester transfer protein (CETP); a bile acid sequestrant; a fibrate; a phytosterol; or a PCSK9 inhibitor.
27. The method, drug, or other lipid lowering drug of item 26, wherein
   the cholesterol absorption inhibitor is selected from ezetimibe and SCH-48461;
   the cholesteryl ester transfer protein (CETP) inhibitor is selected from anacetrapib, evacetrapib, and dalcetrapib;
   the bile acid sequestrant is selected from colesevelam, cholestyramine and colestipol;
   the fibrate is selected from fenofibrate, gemfibrozil, clofibrate, and bezafibrate; and
   the PCSK9 inhibitor is selected from a PCSK9 specific antibody, an siRNA, and a peptidomimetic.
28. The method of any one of items 11 to 15, 17, 23, or 24, the drug of any one of items 16, 23, or 24, or the statin, other lipid lowering drug, or modulator of lipid/lipid concentration ratios of item 25, wherein the statin is selected from the group consisting of atorvastatin, cerivastatin, fluvastatin, fluvastatin XL, lovastatin, pitavastatin, pravastatin, rosuvastatin and simvastatin.
29. The method of any one of items 11 to 15, 17, 23, or 24, the drug of any one of items 16, 23, or 24, or the statin, other lipid lowering drug, or modulator of lipid/lipid concentration ratios of item 25, wherein the modulator of lipid/lipid concentration ratios is selected from a small molecule, an antibody, an antisense RNA, a small interfering RNA (siRNA), and a natural or modified lipid, preferably a lipid of any one of the lipid/lipid concentration ratios referred to in items 1 to 4 or 7.
30. The method of any one of items 1 to 15, 17, 23, 24, and 26-29, the drug of any one of items 16, 23, 24 and 26-29, or the statin, other lipid lowering drug, or modulator of lipid/lipid concentration ratios of any one of items 25-29, wherein the control is from a healthy individual,
   a generalized population of healthy individuals,
   a CAD patient that has remained free of any major CVD complications, or
   a group of CAD patients that has remained free of any major CVD complications.
31. The method, the drug, the statin, the other lipid lowering drug, or the modulator of lipid/lipid concentration ratios of item 30, wherein the control is a non-HDL sample.
32. The method, the drug, the statin, the other lipid lowering drug, or the modulator of lipid/lipid concentration ratios of item 30 or 31, wherein the control is
   an LDL sample,
   a very-low density lipoprotein (VLDL) sample, or
   an intermediate-density lipoprotein (IDL) sample, or combinations thereof.
33. The method, the drug, the statin, the other lipid lowering drug, or the modulator of lipid/lipid concentration ratios of item 30 or 31, wherein the control is an LDL sample.
34. The method, the drug, the use, the kit, the antibody, the statin, the other lipid lowering drug, or the modulator of lipid/lipid concentration ratios of any one of the preceding items, wherein the one or more complications of atherosclerosis or CVD to be prevented or treated is selected from myocardial infarction (MI), acute myocardial infarction (AMI), angina pectoris, transient ischemic attack (TIA), and stroke.
35. The method, the drug, the use, the kit, the antibody, the statin, the other lipid lowering drug, or the modulator of lipid/lipid concentration ratios of any one of the preceding items, wherein the one or more complications of atherosclerosis or CVD to be prevented is death.

### REFERENCES

1. Parish, S. et al. Lipids and lipoproteins and risk of different vascular events in the MRC/BHF Heart Protection Study. Circulation 125, 2469-78 (2012).
2. St∪hlman, M. et al. Proteomics and lipids of lipoproteins isolated at low salt concentrations in D20/sucrose or in KBr. Journal of lipid research 49, 481-90 (2008).
3. Jung, H. R. et al. High throughput quantitative molecular lipidomics. Biochimica et biophysica acta 1811, 925-34 (2011).
4. Deckelbaum, R. J., Granot, E., Oschry, Y., Rose, L. & Eisenberg, S. Plasma triglyceride determines structure-composition in low and high density lipoproteins. Arteriosclerosis, Thrombosis, and Vascular Biology 4, 225-231 (1984).
5. Burstein, M., Scholnick, H. R., & Morfin, R. (1970). Rapid method for the isolation of lipoproteins from human serum by precipitation with polyanions, 11.
6. Dallinga-Thie, G. M., Schneijderberg, V. L. M., & Toll, A. Van. (1986). Identification and characterization of rat serum lipoprotein subclasses. Isolation by chromatography on agarose columns and sequential immunoprecipitation, 27, 1035-1043.
7. Wiesner, P., Leidl, K., Boettcher, A., Schmitz, G., & Liebisch, G. (2009). Lipid profiling of FPLC-separated lipoprotein fractions by electrospray ionization tandem mass spectrometry. Journal of lipid research, 50(3), 574-85. doi:10.1194/jlr.D800028-JLR200.
8. Sullards, M. C., et al., Structure-specific, quantitative methods for analysis of sphingolipids by liquid chromatography-tandem mass spectrometry: “inside-out” sphingolipidomics. Methods Enzymol 2007.
9. Fong, B., et al., Liquid chromatography-high-resolution mass spectrometry for quantitative analysis of gangliosides. Lipids, 2009. 44(9): p. 867-74.
10. St∪hlman, M., et al., High-throughput shotgun lipidomics by quadrupole time-of-flight mass spectrometry. J Chromatogr B Analyt Technol Biomed Life Sci, 2009.
11. Ekroos, K., et al., Quantitative profiling of phospholipids by multiple precursor ion scanning on a hybrid quadrupole time-of-flight mass spectrometer. Anal Chem, 2002. 74(5): p. 941-9.
12. Ekroos, K., et al., Charting molecular composition of phosphatidylcholines by fatty acid scanning and ion trap MS3 fragmentation. J Lipid Res, 2003. 44(11): p. 2181-92.
13. Ejsing, C. S., et al., Automated identification and quantification of glycerophospholipid molecular species by multiple precursor ion scanning. Anal Chem, 2006. 78(17): p. 6202-14.

Those skilled in the art will recognize, or be able to ascertain using no more than routine experimentation, numerous equivalents to the specific embodiments described herein both in the Examples and in the body of the entire patent description. Such equivalents are considered to be within the scope of this invention and are intended to be encompassed by the following claims or the items listed above. All patents, patent applications, and published references cited herein are hereby incorporated by reference in their entirety.

1-51. (canceled)

52. A method of obtaining data for use in determining whether a subject is at risk to develop, or is suffering from atherosclerosis or cardiovascular disease (CVD) and/or one or more of its complications, comprising
(a) determining in a non-HDL sample from said subject a ceramide/TAG concentration ratio;
(b) determining in a non-HDL sample from said subject a CE/TAG concentration ratio;
(c) determining in a non-HDL sample from said subject a LPE/TAG concentration ratio; or
(d) determining in a non-HDL sample from said subject a ceramide/CE concentration ratio.

53. A method for determining whether a subject is at risk to develop, or is suffering from atherosclerosis or cardiovascular disease (CVD) and/or one or more of its complications, said method comprising
(a) determining in a non-HDL sample from said subject a ceramide/TAG concentration ratio, wherein a decreased ratio in said sample, when compared to a control, is indicative of said subject suffering from or having an increased risk of developing atherosclerosis or CVD and/or one or more of its complications;
(b) determining in a non-HDL sample from said subject a CE/TAG concentration ratio, wherein a decreased ratio in said sample, when compared to a control, is indicative of said subject suffering from or having an increased risk of developing atherosclerosis or CVD and/or one or more of its complications;
(c) determining in a non-HDL sample from said subject a LPE/TAG concentration ratio, wherein a decreased ratio in said sample, when compared to a control, is indicative of said subject suffering from or having an increased risk of developing atherosclerosis or CVD and/or one or more of its complications; or
(d) determining in a non-HDL sample from said subject a ceramide/CE concentration ratio, wherein a decreased ratio in said sample, when compared to a control, is indicative of said subject suffering from or having an increased risk of developing atherosclerosis or CVD and/or one or more of its complications.

54. A method for evaluating the effectiveness of a treatment of atherosclerosis or CVD and/or one or more of its complications in a subject, comprising
(a) determining in a non-HDL sample from said subject a ceramide/TAG concentration ratio, wherein an increased ratio in said sample, when compared to a control, is indicative of the effectiveness of said treatment;
(b) determining in a non-HDL sample from said subject a CE/TAG concentration ratio, wherein a increased ratio in said sample, when compared to a control, is indicative of the effectiveness of said treatment;
(c) determining in a non-HDL sample from said subject a LPE/TAG concentration ratio, wherein an increased ratio in said sample, when compared to a control, is indicative of the effectiveness of said treatment; or
(d) determining in a non-HDL sample from said subject a ceramide/CE concentration ratio, wherein an increased ratio in said sample, when compared to a control, is indicative of the effectiveness of said treatment.

55. A method of choosing an appropriate treatment of atherosclerosis or CVD and/or one or more of its complications in a subject, comprising
(a) determining in a non-HDL sample from said subject a ceramide/TAG concentration ratio, wherein a decreased ratio in said sample, when compared to a control, is indicative of said subject being in need of treatment or a change in, or supplementation of, an already administered treatment;
(b) determining in a non-HDL sample from said subject a CE/TAG concentration ratio, wherein a decreased ratio in said sample, when compared to a control, is indicative of said subject being in need of treatment or a change in, or supplementation of, an already administered treatment;
(c) determining in a non-HDL sample from said subject a LPE/TAG concentration ratio, wherein a decreased ratio in said sample, when compared to a control, is indicative of said subject being in need of treatment or a change in, or supplementation of, an already administered treatment; or
(d) determining in a non-HDL sample from said subject a ceramide/CE concentration ratio, wherein a decreased ratio in said sample, when compared to a control, is indicative of said subject being in need of treatment or a change in, or supplementation of, an already administered treatment.

56. The method of claim 52, wherein the method is a computer-implemented method.

57. The method of claim 56, further comprising
(e) obtaining by at least one processor information reflecting the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample;
(f) determining by at least one processor the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample; and
(g) outputting in user readable format the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample.

58. The method of claim 57, further comprising
(h) determining by at least one processor a percentage difference between a control and the ceramide/TAG concentration ratio in the non-HDL sample, the CE/TAG concentration ratio in the non-HDL sample, the LPE/TAG concentration ratio in the non-HDL sample, or the ceramide/CE concentration in the non-HDL sample; and
(i) outputting in user readable format the percentage difference obtained in the determining step (h).

59. The method of claim 58, further comprising determining whether a subject is at risk to develop, or is suffering from atherosclerosis or cardiovascular disease (CVD) and/or one or more of its complications based on the percentage difference obtained in the outputting step.

60. The method of claim 54, further comprising after the determining step, changing, supplementing, or keeping the same an already administered treatment in said subject based on the ceramide/TAG concentration ratio, CE/TAG concentration ratio, LPE/TAG concentration ratio, or ceramide/CE concentration ratio obtained in the determining step.

61. The method of claim 55, further comprising after the determining step, treating said subject based on the ceramide/TAG concentration ratio, CE/TAG concentration ratio, LPE/TAG concentration ratio, or ceramide/CE concentration ratio obtained in the determining step.

62. The method of claim 52, wherein
(a) the ceramide/TAG concentration ratio is selected from any of the ceramide/TAG concentration ratios referred to in Table 1;
(b) the CE/TAG concentration ratio is selected from any of the CE/TAG concentration ratios referred to in Table 2;
(c) the LPE/TAG concentration ratio is selected from any of the LPE/TAG concentration ratios referred to in Table 3; or
(d) the ceramide/CE concentration ratio is selected from any of the ceramide/CE concentration ratios referred to in Table 4.

63. The method of claim 52, wherein determining the lipid/lipid concentration ratio(s) is done using an assay.

64. The method of claim 54, wherein said treatment is a lipid modifying treatment.

65. The method of claim 52, wherein
(a) the ceramide/TAG concentration ratio is selected from the group consisting of:
Glc/GalCer(d18:1/22:0)/TAG 50:1 total (16:0/16:0/18:1);
Cer(d18:1/22:0)/TAG 50:1 total (16:0/16:0/18:1);
GM3-d18:1/24:0/TAG 54:2 total (18:0/18:1/18:1);
Cer(d18:1/18:0)/TAG 50:2 total (14:0/18:1/18:1)(16:0/16:0/18:2)(16:0/16:1/18:1);
Cer(d18:1/18:0)/TAG 50:3 total (14:0/18:1/18:2)(16:0/16:1/18:2)(16:1/16:1/18:1);
Cer(d18:1/18:0)/TAG 52:3 total (16:0/18:1/18:2)(16:1/18:1/18:1); and
Cer(d18:1/18:0)/TAG 56:6 total (18:1/18:1/20:4);
(b) the CE/TAG concentration ratio is selected from the group consisting of:
CE 22:6/TAG 50:1 total (16:0/16:0/18:1);
CE 16:0/TAG 50:1 total (16:0/16:0/18:1);
CE 22:6/TAG 50:2 total (14:0/18:1/18:1)(16:0/16:0/18:2)(16:0/16:1/18:1);
CE 16:0/TAG 50:2 total (14:0/18:1/18:1)(16:0/16:0/18:2)(16:0/16:1/18:1);
CE 18:1/TAG 50:2 total (14:0/18:1/18:1)(16:0/16:0/18:2)(16:0/16:1/18:1);
CE 18:2/TAG 54:2 total (18:0/18:1/18:1);
CE 18:2/Total TAG;
CE 22:6/TAG 50:3 total (14:0/18:1/18:2)(16:0/16:1/18:2)(16:1/16:1/18:1); and
CE 22:6/TAG 52:3 total (16:0/18:1/18:2)(16:1/18:1/18:1);
(c) the LPE/TAG concentration ratio is selected from the group consisting of:
LPE 18:0/TAG 50:1 total (16:0/16:0/18:1);
LPE 18:0/TAG 50:2 total (14:0/18:1/18:1)(16:0/16:0/18:2)(16:0/16:1/18:1); and
LPE 16:0/TAG 50:2 total (14:0/18:1/18:1)(16:0/16:0/18:2)(16:0/16:1/18:1); or
(d) the ceramide/CE concentration ratio is selected from the group consisting of:
Cer(d18:1/18:0)/CE 22:0;
Cer(d18:1/20:0)/CE 22:0; and
Cer(d18:1/22:0)/CE 22:0.

66. The method of claim 52, comprising determining at least 2, at least 3, at least 4, at least 5, at least 6, at least 7, or at least 8 of the lipid/lipid concentration ratios referred to therein, or combinations thereof.

67. The method of claim 52, wherein
(a) said CVD is characterized by coronary artery disease, peripheral artery disease, a stroke and/or CVD death; and/or
(b) said CVD is atherosclerosis-induced; and/or
(c) said subject has atherosclerosis; or
(d) said subject does not have atherosclerosis.

68. The method of claim 52, wherein
(a) the method further comprises determining the serum level of total cholesterol, low-density lipoprotein cholesterol (LDL-C), high-density lipoprotein cholesterol (HDL-C), Apolipoprotein B (ApoB) and/or Apolipoprotein C-III (ApoC-III) in a sample from said subject; and/or
(b) the subject does not have elevated serum levels of one or more of total cholesterol, low-density lipoprotein cholesterol (LDL-C), Apolipoprotein C-III (ApoC-III) or Apolipoprotein B (ApoB), or a decreased serum level of HDL-cholesterol (HDL-C).

69. The method of claim 52, wherein said subject
(a) is being or has been treated with a statin, another lipid lowering drug, and/or a modulator of lipid/lipid concentration ratios; or
(b) has not yet undergone statin therapy, therapy with another lipid lowering drug, and/or therapy with a modulator of lipid/lipid concentration ratios.

70. The method of claim 52, wherein the non-HDL sample is a LDL sample, a very-low density lipoprotein (VLDL) sample, or an intermediate-density lipoprotein (IDL) sample, or combinations thereof.

71. The method of claim 52, wherein the non-HDL sample is an LDL sample.

72. The method of claim 52, wherein the lipid/lipid concentration ratio is determined by using mass spectrometry, nuclear magnetic resonance spectroscopy, fluorescence spectroscopy or dual polarisation interferometry, a high performance separation method such as HPLC, UHPLC or UPLC, an immunoassay such as an ELISA and/or an assay with a binding moiety capable of specifically binding the analyte.

73. The method of claim 52, wherein the method is for:
(a) determining a risk of said patient to develop CVD;
(b) determining early warning signs of CVD in said patient;
(c) determining or predicting the occurrence of atherosclerosis in a patient; and/or
(d) predicting and/or diagnosing CVD and/or CVD complications including predicting and/or diagnosing myocardial infarction (MI), angina pectoris, transient ischemic attack (TIA) and stroke, or predicting death.

74. A method for determining whether a subject is at risk to develop, or is suffering from atherosclerosis or CVD and/or one or more of its complications, said method comprising:
(a) determining in a non-HDL sample from said subject a ceramide/TAG concentration ratio using an antibody against any one of the lipids in any one of the ceramide/TAG concentration ratios referred to in claim 52, wherein a decreased ceramide/TAG concentration ratio in said sample, when compared to a control, is indicative of said subject suffering from or having an increased risk of developing atherosclerosis or CVD and/or one or more of its complications;
(b) determining in a non-HDL sample from said subject a CE/TAG concentration ratio using an antibody against any one of the lipids in any one of the CE/TAG concentration ratios referred to in claim 52, wherein a decreased CE/TAG concentration ratio in said sample, when compared to a control, is indicative of said subject suffering from or having an increased risk of developing atherosclerosis or CVD and/or one or more of its complications;
(c) determining in a non-HDL sample from said subject a LPE/TAG concentration ratio using an antibody against any one of the lipids in any one of the LPE/TAG concentration ratios referred to in claim 52, wherein a decreased LPE/TAG concentration ratio in said sample, when compared to a control, is indicative of said subject suffering from or having an increased risk of developing atherosclerosis or CVD and/or one or more of its complications; or
(d) determining in a non-HDL sample from said subject a ceramide/CE concentration ratio using an antibody against any one of the lipids in any one of the ceramide/CE concentration ratios referred to in claim 52, wherein a decreased ceramide/CE concentration ratio in said sample, when compared to a control, is indicative of said subject suffering from or having an increased risk of developing atherosclerosis or CVD and/or one or more of its complications.

75. The method of claim 52, wherein the subject is at risk to develop or has suffered from one or more CVD complications such as acute myocardial infarction and/or is at risk of cardiovascular death.

76. The method of claim 52, wherein the subject has suffered from a cardiovascular disease.
